SEQUENCE LISTING (455 sequences)

SEQ ID NO: 1 - protein, 15 aa, Artificial Sequence
  Description of Artificial Sequence Synthetic peptide
MWQPRRPWPRVPWRW

SEQ ID NO: 2 - protein, 15 aa, Artificial Sequence
  Description of Artificial Sequence Synthetic peptide
LRLLHRRQKRIIGGK

SEQ ID NO: 3 - protein, 15 aa, Artificial Sequence
  Description of Artificial Sequence Synthetic peptide
RQHGLRHFYNRRRRS

SEQ ID NO: 4 - protein, 20 aa, Artificial Sequence
  Description of Artificial Sequence Synthetic peptide
KLWKKKELLQRAEKKKKIKK

SEQ ID NO: 5 - protein, 20 aa, Artificial Sequence
  Description of Artificial Sequence Synthetic peptide
MPKFKQRRRKLKAKAERLFK

SEQ ID NO: 6 - protein, 20 aa, Artificial Sequence
  Description of Artificial Sequence Synthetic peptide
FVFPRLRDFTLAMAARKASR

SEQ ID NO: 7 - protein, 15 aa, Artificial Sequence
  Description of Artificial Sequence Synthetic peptide
YLKFIPLKRAIWLIK

SEQ ID NO: 8 - protein, 20 aa, Artificial Sequence
  Description of Artificial Sequence Synthetic peptide
IKRKRPFVLKKKRGRKRRRI

SEQ ID NO: 9 - protein, 20 aa, Artificial Sequence
  Description of Artificial Sequence Synthetic peptide
RTTRRWKRWFKFRKRKGEKR

SEQ ID NO: 10 - protein, 15 aa, Artificial Sequence
  Description of Artificial Sequence Synthetic peptide
MVLKFFRWLFRLLFR

SEQ ID NO: 11 - protein, 15 aa, Artificial Sequence
  Description of Artificial Sequence Synthetic peptide
RLWEFYKLYKRRHRV

SEQ ID NO: 12 - protein, 15 aa, Artificial Sequence
  Description of Artificial Sequence Synthetic peptide
KVFSPKKKMEFFLLF

SEQ ID NO: 13 - protein, 15 aa, Artificial Sequence
  Description of Artificial Sequence Synthetic peptide
VKIWFQNRRVRWRKR

SEQ ID NO: 14 - protein, 15 aa, Artificial Sequence
  Description of Artificial Sequence Synthetic peptide
MRMIRFRKKIPYLRY

SEQ ID NO: 15 - protein, 15 aa, Artificial Sequence
  Description of Artificial Sequence Synthetic peptide
PKWTRPLLPFWKRYL

SEQ ID NO: 16 - protein, 15 aa, Artificial Sequence
  Description of Artificial Sequence Synthetic peptide
RWFAFKMMMAKKWAK

SEQ ID NO: 17 - protein, 15 aa, Artificial Sequence
  Description of Artificial Sequence Synthetic peptide
SKIVRVIFRYAKWLF

SEQ ID NO: 18 - protein, 15 aa, Artificial Sequence
  Description of Artificial Sequence Synthetic peptide
KFFKLKHFILNILKQ

SEQ ID NO: 19 - protein, 15 aa, Artificial Sequence
  Description of Artificial Sequence Synthetic peptide
LLPQWPRIRHIKLLR

SEQ ID NO: 20 - protein, 12 aa, Artificial Sequence
  Description of Artificial Sequence Synthetic peptide
AARLWFFRLWRR

SEQ ID NO: 21 - protein, 12 aa, Artificial Sequence
  Description of Artificial Sequence Synthetic peptide
AFFILKWKLWKK

SEQ ID NO: 22 - protein, 12 aa, Artificial Sequence
  Description of Artificial Sequence Synthetic peptide
AFLFRRFYDRRF

SEQ ID NO: 23 - protein, 12 aa, Artificial Sequence
  Description of Artificial Sequence Synthetic peptide
AFRFIKRLWRLV

SEQ ID NO: 24 - protein, 12 aa, Artificial Sequence
  Description of Artificial Sequence Synthetic peptide
AIVLYFFCRRRL

SEQ ID NO: 25 - protein, 12 aa, Artificial Sequence
  Description of Artificial Sequence Synthetic peptide
ALFFAWKRIYRP

SEQ ID NO: 26 - protein, 12 aa, Artificial Sequence
  Description of Artificial Sequence Synthetic peptide
ALFFAWRRIVRP

SEQ ID NO: 27 - protein, 12 aa, Artificial Sequence
  Description of Artificial Sequence Synthetic peptide
ALFFAWRRIYRP

SEQ ID NO: 28 - protein, 12 aa, Artificial Sequence
  Description of Artificial Sequence Synthetic peptide
ALICFLIFWRRR

SEQ ID NO: 29 - protein, 12 aa, Artificial Sequence
  Description of Artificial Sequence Synthetic peptide
AWAVMARWFWRR

SEQ ID NO: 30 - protein, 12 aa, Artificial Sequence
  Description of Artificial Sequence Synthetic peptide
AWRFLGRVWRLV

SEQ ID NO: 31 - protein, 12 aa, Artificial Sequence
  Description of Artificial Sequence Synthetic peptide
AWRLRKNFFYFY

SEQ ID NO: 32 - protein, 12 aa, Artificial Sequence
  Description of Artificial Sequence Synthetic peptide
CRFIMRCWLCWK

SEQ ID NO: 33 - protein, 12 aa, Artificial Sequence
  Description of Artificial Sequence Synthetic peptide
CRLLWIFRRRWR

SEQ ID NO: 34 - protein, 12 aa, Artificial Sequence
  Description of Artificial Sequence Synthetic peptide
FAFRFAFKRWLT

SEQ ID NO: 35 - protein, 12 aa, Artificial Sequence
  Description of Artificial Sequence Synthetic peptide
FALILIFRRKWK

SEQ ID NO: 36 - protein, 12 aa, Artificial Sequence
  Description of Artificial Sequence Synthetic peptide
FCGFLWFFKYKR

SEQ ID NO: 37 - protein, 12 aa, Artificial Sequence
  Description of Artificial Sequence Synthetic peptide
FFALRYIMRLRA

SEQ ID NO: 38 - protein, 12 aa, Artificial Sequence
  Description of Artificial Sequence Synthetic peptide
FFCFFRKRWKVL

SEQ ID NO: 39 - protein, 12 aa, Artificial Sequence
  Description of Artificial Sequence Synthetic peptide
FFCWAWLPRRRR

SEQ ID NO: 40 - protein, 12 aa, Artificial Sequence
  Description of Artificial Sequence Synthetic peptide
FFFFKCRRWLCF

SEQ ID NO: 41 - protein, 12 aa, Artificial Sequence
  Description of Artificial Sequence Synthetic peptide
FFFLRRFERGFW

SEQ ID NO: 42 - protein, 12 aa, Artificial Sequence
  Description of Artificial Sequence Synthetic peptide
FFFVARDLWKWR

SEQ ID NO: 43 - protein, 12 aa, Artificial Sequence
  Description of Artificial Sequence Synthetic peptide
FFFWKIRPQIAR

SEQ ID NO: 44 - protein, 12 aa, Artificial Sequence
  Description of Artificial Sequence Synthetic peptide
FFFWKIYPQIRK

SEQ ID NO: 45 - protein, 12 aa, Artificial Sequence
  Description of Artificial Sequence Synthetic peptide
FFILKRLNLLRI

SEQ ID NO: 46 - protein, 12 aa, Artificial Sequence
  Description of Artificial Sequence Synthetic peptide
FFIRLFRKIGWG

SEQ ID NO: 47 - protein, 12 aa, Artificial Sequence
  Description of Artificial Sequence Synthetic peptide
FFIRRLRLLKLE

SEQ ID NO: 48 - protein, 12 aa, Artificial Sequence
  Description of Artificial Sequence Synthetic peptide
FFKRLPKWRLGI

SEQ ID NO: 49 - protein, 12 aa, Artificial Sequence
  Description of Artificial Sequence Synthetic peptide
FFLKRKMKEFLF

SEQ ID NO: 50 - protein, 12 aa, Artificial Sequence
  Description of Artificial Sequence Synthetic peptide
FFLQMAVYRRRF

SEQ ID NO: 51 - protein, 12 aa, Artificial Sequence
  Description of Artificial Sequence Synthetic peptide
FFMYYFLWKKNR

SEQ ID NO: 52 - protein, 12 aa, Artificial Sequence
  Description of Artificial Sequence Synthetic peptide
FFRFLLRKLYVF

SEQ ID NO: 53 - protein, 12 aa, Artificial Sequence
  Description of Artificial Sequence Synthetic peptide
FFRLFRVLRLVK

SEQ ID NO: 54 - protein, 12 aa, Artificial Sequence
  Description of Artificial Sequence Synthetic peptide
FFRLFRVMRLIK

SEQ ID NO: 55 - protein, 12 aa, Artificial Sequence
  Description of Artificial Sequence Synthetic peptide
FFRLFRVMRLVK

SEQ ID NO: 56 - protein, 12 aa, Artificial Sequence
  Description of Artificial Sequence Synthetic peptide
FFRLFRVMRLVK

SEQ ID NO: 57 - protein, 12 aa, Artificial Sequence
  Description of Artificial Sequence Synthetic peptide
FFRYILKRYFNY

SEQ ID NO: 58 - protein, 12 aa, Artificial Sequence
  Description of Artificial Sequence Synthetic peptide
FGAFLKRMRRLF

SEQ ID NO: 59 - protein, 12 aa, Artificial Sequence
  Description of Artificial Sequence Synthetic peptide
FGRFYRGRIWYL

SEQ ID NO: 60 - protein, 12 aa, Artificial Sequence
  Description of Artificial Sequence Synthetic peptide
FIGILFRILRKR

SEQ ID NO: 61 - protein, 12 aa, Artificial Sequence
  Description of Artificial Sequence Synthetic peptide
FILMKKWKFHLV

SEQ ID NO: 62 - protein, 12 aa, Artificial Sequence
  Description of Artificial Sequence Synthetic peptide
FILWIKRIMRLK

SEQ ID NO: 63 - protein, 12 aa, Artificial Sequence
  Description of Artificial Sequence Synthetic peptide
FIRRIFRRLPTF

SEQ ID NO: 64 - protein, 12 aa, Artificial Sequence
  Description of Artificial Sequence Synthetic peptide
FIRRIFRRLPTF

SEQ ID NO: 65 - protein, 12 aa, Artificial Sequence
  Description of Artificial Sequence Synthetic peptide
FITWLKLRLRYI

SEQ ID NO: 66 - protein, 12 aa, Artificial Sequence
  Description of Artificial Sequence Synthetic peptide
FKAFFIRRYFVF

SEQ ID NO: 67 - protein, 12 aa, Artificial Sequence
  Description of Artificial Sequence Synthetic peptide
FKFFFRRNPYFR

SEQ ID NO: 68 - protein, 12 aa, Artificial Sequence
  Description of Artificial Sequence Synthetic peptide
FKKLIPWFSFRM

SEQ ID NO: 69 - protein, 12 aa, Artificial Sequence
  Description of Artificial Sequence Synthetic peptide
FKRILLNILYRF

SEQ ID NO: 70 - protein, 12 aa, Artificial Sequence
  Description of Artificial Sequence Synthetic peptide
FKRIPWFIKKRI

SEQ ID NO: 71 - protein, 12 aa, Artificial Sequence
  Description of Artificial Sequence Synthetic peptide
FKVGLWKRYFIL

SEQ ID NO: 72 - protein, 12 aa, Artificial Sequence
  Description of Artificial Sequence Synthetic peptide
FLALPLRLRRRI

SEQ ID NO: 73 - protein, 12 aa, Artificial Sequence
  Description of Artificial Sequence Synthetic peptide
FLAMPLRWRLKI

SEQ ID NO: 74 - protein, 12 aa, Artificial Sequence
  Description of Artificial Sequence Synthetic peptide
FLFFKGKKAYWF

SEQ ID NO: 75 - protein, 12 aa, Artificial Sequence
  Description of Artificial Sequence Synthetic peptide
FLFLKWRRIRKF

SEQ ID NO: 76 - protein, 12 aa, Artificial Sequence
  Description of Artificial Sequence Synthetic peptide
FLFPRRRVKRLI

SEQ ID NO: 77 - protein, 12 aa, Artificial Sequence
  Description of Artificial Sequence Synthetic peptide
FLFRVFRRRLQA

SEQ ID NO: 78 - protein, 12 aa, Artificial Sequence
  Description of Artificial Sequence Synthetic peptide
FLILRIKLKRIY

SEQ ID NO: 79 - protein, 12 aa, Artificial Sequence
  Description of Artificial Sequence Synthetic peptide
FLIVRMRELLWR

SEQ ID NO: 80 - protein, 12 aa, Artificial Sequence
  Description of Artificial Sequence Synthetic peptide
FLIYKFKRKIPW

SEQ ID NO: 81 - protein, 12 aa, Artificial Sequence
  Description of Artificial Sequence Synthetic peptide
FLKFPFLKKYRI

SEQ ID NO: 82 - protein, 12 aa, Artificial Sequence
  Description of Artificial Sequence Synthetic peptide
FLKLYVLIKWCR

SEQ ID NO: 83 - protein, 12 aa, Artificial Sequence
  Description of Artificial Sequence Synthetic peptide
FLKRYLLFQLRW

SEQ ID NO: 84 - protein, 12 aa, Artificial Sequence
  Description of Artificial Sequence Synthetic peptide
FLKRYLLFQLRW

SEQ ID NO: 85 - protein, 12 aa, Artificial Sequence
  Description of Artificial Sequence Synthetic peptide
FLLAAYFFRFRK

SEQ ID NO: 86 - protein, 12 aa, Artificial Sequence
  Description of Artificial Sequence Synthetic peptide
FLLCYWKACWRR

SEQ ID NO: 87 - protein, 12 aa, Artificial Sequence
  Description of Artificial Sequence Synthetic peptide
FLLIRRVLRYYL

SEQ ID NO: 88 - protein, 12 aa, Artificial Sequence
  Description of Artificial Sequence Synthetic peptide
FLLLKVFYRVLR

SEQ ID NO: 89 - protein, 12 aa, Artificial Sequence
  Description of Artificial Sequence Synthetic peptide
FLLLLLFLKRKK

SEQ ID NO: 90 - protein, 12 aa, Artificial Sequence
  Description of Artificial Sequence Synthetic peptide
FLLPWRRWWQQR

SEQ ID NO: 91 - protein, 12 aa, Artificial Sequence
  Description of Artificial Sequence Synthetic peptide
FLLRRGIYRAWM

SEQ ID NO: 92 - protein, 12 aa, Artificial Sequence
  Description of Artificial Sequence Synthetic peptide
FLLSMRYFFRPK

SEQ ID NO: 93 - protein, 12 aa, Artificial Sequence
  Description of Artificial Sequence Synthetic peptide
FLMKKWKYFLIH

SEQ ID NO: 94 - protein, 12 aa, Artificial Sequence
  Description of Artificial Sequence Synthetic peptide
FLMLLRRFRSRY

SEQ ID NO: 95 - protein, 12 aa, Artificial Sequence
  Description of Artificial Sequence Synthetic peptide
FLRFVLRKLYVF

SEQ ID NO: 96 - protein, 12 aa, Artificial Sequence
  Description of Artificial Sequence Synthetic peptide
FLRFVLRKLYVF

SEQ ID NO: 97 - protein, 12 aa, Artificial Sequence
  Description of Artificial Sequence Synthetic peptide
FLRLFRAARLIK

SEQ ID NO: 98 - protein, 12 aa, Artificial Sequence
  Description of Artificial Sequence Synthetic peptide
FLRYLSWRFWKF

SEQ ID NO: 99 - protein, 12 aa, Artificial Sequence
  Description of Artificial Sequence Synthetic peptide
FLTLPLFIRRRI

SEQ ID NO: 100 - protein, 12 aa, Artificial Sequence
  Description of Artificial Sequence Synthetic peptide
FLWIPLRLRLRI

SEQ ID NO: 101 - protein, 12 aa, Artificial Sequence
  Description of Artificial Sequence Synthetic peptide
FLWLPLRFRLRI

SEQ ID NO: 102 - protein, 12 aa, Artificial Sequence
  Description of Artificial Sequence Synthetic peptide
FLYFRRTPRPLF

SEQ ID NO: 103 - protein, 12 aa, Artificial Sequence
  Description of Artificial Sequence Synthetic peptide
FMFLFFLWRKPR

SEQ ID NO: 104 - protein, 12 aa, Artificial Sequence
  Description of Artificial Sequence Synthetic peptide
FMWVRWTLPRFR

SEQ ID NO: 105 - protein, 12 aa, Artificial Sequence
  Description of Artificial Sequence Synthetic peptide
FPWRKFPRYLKV

SEQ ID NO: 106 - protein, 12 aa, Artificial Sequence
  Description of Artificial Sequence Synthetic peptide
FPWSFRLKRLLY

SEQ ID NO: 107 - protein, 12 aa, Artificial Sequence
  Description of Artificial Sequence Synthetic peptide
FQLFFRRFLRLS

SEQ ID NO: 108 - protein, 12 aa, Artificial Sequence
  Description of Artificial Sequence Synthetic peptide
FRFRFWRFGKWV

SEQ ID NO: 109 - protein, 12 aa, Artificial Sequence
  Description of Artificial Sequence Synthetic peptide
FRGLFRFLRFIE

SEQ ID NO: 110 - protein, 12 aa, Artificial Sequence
  Description of Artificial Sequence Synthetic peptide
FRKFPWYKVPIY

SEQ ID NO: 111 - protein, 12 aa, Artificial Sequence
  Description of Artificial Sequence Synthetic peptide
FRKRMMLAYRFR

SEQ ID NO: 112 - protein, 12 aa, Artificial Sequence
  Description of Artificial Sequence Synthetic peptide
FRMKLRNLFIKF

SEQ ID NO: 113 - protein, 12 aa, Artificial Sequence
  Description of Artificial Sequence Synthetic peptide
FRPLAPRPWRWL

SEQ ID NO: 114 - protein, 12 aa, Artificial Sequence
  Description of Artificial Sequence Synthetic peptide
FRRFFTRQFYLW

SEQ ID NO: 115 - protein, 12 aa, Artificial Sequence
  Description of Artificial Sequence Synthetic peptide
FRRFFYRLLSLK

SEQ ID NO: 116 - protein, 12 aa, Artificial Sequence
  Description of Artificial Sequence Synthetic peptide
FRRFVWNFFRLE

SEQ ID NO: 117 - protein, 12 aa, Artificial Sequence
  Description of Artificial Sequence Synthetic peptide
FRRFVWNFFRLE

SEQ ID NO: 118 - protein, 12 aa, Artificial Sequence
  Description of Artificial Sequence Synthetic peptide
FRRLPLRLRLKI

SEQ ID NO: 119 - protein, 12 aa, Artificial Sequence
  Description of Artificial Sequence Synthetic peptide
FRRMHLRITFFR

SEQ ID NO: 120 - protein, 12 aa, Artificial Sequence
  Description of Artificial Sequence Synthetic peptide
FRSRLFYLFHRF

SEQ ID NO: 121 - protein, 12 aa, Artificial Sequence
  Description of Artificial Sequence Synthetic peptide
FRTFFRLPKWMW

SEQ ID NO: 122 - protein, 12 aa, Artificial Sequence
  Description of Artificial Sequence Synthetic peptide
FVFFFRWRGNYK

SEQ ID NO: 123 - protein, 12 aa, Artificial Sequence
  Description of Artificial Sequence Synthetic peptide
FVFKGRWFWRVR

SEQ ID NO: 124 - protein, 12 aa, Artificial Sequence
  Description of Artificial Sequence Synthetic peptide
FVIIMMWRRKPK

SEQ ID NO: 125 - protein, 12 aa, Artificial Sequence
  Description of Artificial Sequence Synthetic peptide
FVIIMVWRRKPR

SEQ ID NO: 126 - protein, 12 aa, Artificial Sequence
  Description of Artificial Sequence Synthetic peptide
FVIPRPRIPKWW

SEQ ID NO: 127 - protein, 12 aa, Artificial Sequence
  Description of Artificial Sequence Synthetic peptide
FWKRYHKTFIFF

SEQ ID NO: 128 - protein, 12 aa, Artificial Sequence
  Description of Artificial Sequence Synthetic peptide
FYFRPFRLDWFR

SEQ ID NO: 129 - protein, 12 aa, Artificial Sequence
  Description of Artificial Sequence Synthetic peptide
FYLIIRRKPLFY

SEQ ID NO: 130 - protein, 12 aa, Artificial Sequence
  Description of Artificial Sequence Synthetic peptide
GGRWFRWFGRRF

SEQ ID NO: 131 - protein, 12 aa, Artificial Sequence
  Description of Artificial Sequence Synthetic peptide
GHFIFKYRRVWW

SEQ ID NO: 132 - protein, 12 aa, Artificial Sequence
  Description of Artificial Sequence Synthetic peptide
GLKYRLFYWLRR

SEQ ID NO: 133 - protein, 12 aa, Artificial Sequence
  Description of Artificial Sequence Synthetic peptide
GYFVFWFRKVRL

SEQ ID NO: 134 - protein, 12 aa, Artificial Sequence
  Description of Artificial Sequence Synthetic peptide
IAMKLYFRRFRP

SEQ ID NO: 135 - protein, 12 aa, Artificial Sequence
  Description of Artificial Sequence Synthetic peptide
IFIKFRRFDLLF

SEQ ID NO: 136 - protein, 12 aa, Artificial Sequence
  Description of Artificial Sequence Synthetic peptide
IFKFWLMWKAKG

SEQ ID NO: 137 - protein, 12 aa, Artificial Sequence
  Description of Artificial Sequence Synthetic peptide
IFKFWLMWKAKG

SEQ ID NO: 138 - protein, 12 aa, Artificial Sequence
  Description of Artificial Sequence Synthetic peptide
IFLKLIKFRIFQ

SEQ ID NO: 139 - protein, 12 aa, Artificial Sequence
  Description of Artificial Sequence Synthetic peptide
IFRIFKLPMVRK

SEQ ID NO: 140 - protein, 12 aa, Artificial Sequence
  Description of Artificial Sequence Synthetic peptide
IFSRYFIRRIRF

SEQ ID NO: 141 - protein, 12 aa, Artificial Sequence
  Description of Artificial Sequence Synthetic peptide
IFYLIRFKIKLM

SEQ ID NO: 142 - protein, 12 aa, Artificial Sequence
  Description of Artificial Sequence Synthetic peptide
IGGFFFLRRFRR

SEQ ID NO: 143 - protein, 12 aa, Artificial Sequence
  Description of Artificial Sequence Synthetic peptide
IILLLLVLRKRR

SEQ ID NO: 144 - protein, 12 aa, Artificial Sequence
  Description of Artificial Sequence Synthetic peptide
IIRFRYFLRRLG

SEQ ID NO: 145 - protein, 12 aa, Artificial Sequence
  Description of Artificial Sequence Synthetic peptide
IKFWRMFFNLYK

SEQ ID NO: 146 - protein, 12 aa, Artificial Sequence
  Description of Artificial Sequence Synthetic peptide
IKKYRYFFCHFW

SEQ ID NO: 147 - protein, 12 aa, Artificial Sequence
  Description of Artificial Sequence Synthetic peptide
ILARPWRAFFKL

SEQ ID NO: 148 - protein, 12 aa, Artificial Sequence
  Description of Artificial Sequence Synthetic peptide
ILFWKFYRVHWK

SEQ ID NO: 149 - protein, 12 aa, Artificial Sequence
  Description of Artificial Sequence Synthetic peptide
ILIVFIKKRIFK

SEQ ID NO: 150 - protein, 12 aa, Artificial Sequence
  Description of Artificial Sequence Synthetic peptide
ILIVFIKKRIFK

SEQ ID NO: 151 - protein, 12 aa, Artificial Sequence
  Description of Artificial Sequence Synthetic peptide
ILLFFYPFYKKR

SEQ ID NO: 152 - protein, 12 aa, Artificial Sequence
  Description of Artificial Sequence Synthetic peptide
ILLLIHFILKRR

SEQ ID NO: 153 - protein, 12 aa, Artificial Sequence
  Description of Artificial Sequence Synthetic peptide
ILPFKRRLEFLW

SEQ ID NO: 154 - protein, 12 aa, Artificial Sequence
  Description of Artificial Sequence Synthetic peptide
ILPYFLTRLFRR

SEQ ID NO: 155 - protein, 12 aa, Artificial Sequence
  Description of Artificial Sequence Synthetic peptide
ILRFRFFRCIKY

SEQ ID NO: 156 - protein, 12 aa, Artificial Sequence
  Description of Artificial Sequence Synthetic peptide
ILRVIRLVRVFR

SEQ ID NO: 157 - protein, 12 aa, Artificial Sequence
  Description of Artificial Sequence Synthetic peptide
IMWLFKMKYARL

SEQ ID NO: 158 - protein, 12 aa, Artificial Sequence
  Description of Artificial Sequence Synthetic peptide
IMYWVLKKFLFK

SEQ ID NO: 159 - protein, 12 aa, Artificial Sequence
  Description of Artificial Sequence Synthetic peptide
IPRPKIPVWWRW

SEQ ID NO: 160 - protein, 12 aa, Artificial Sequence
  Description of Artificial Sequence Synthetic peptide
IRFFLRLINRVR

SEQ ID NO: 161 - protein, 12 aa, Artificial Sequence
  Description of Artificial Sequence Synthetic peptide
IRRWRLRLYLHR

SEQ ID NO: 162 - protein, 12 aa, Artificial Sequence
  Description of Artificial Sequence Synthetic peptide
IVMPLFLRRWKK

SEQ ID NO: 163 - protein, 12 aa, Artificial Sequence
  Description of Artificial Sequence Synthetic peptide
IYGWRKRCLYFF

SEQ ID NO: 164 - protein, 12 aa, Artificial Sequence
  Description of Artificial Sequence Synthetic peptide
IYLKLLVKLYRF

SEQ ID NO: 165 - protein, 12 aa, Artificial Sequence
  Description of Artificial Sequence Synthetic peptide
KFFFLRTRRFAL

SEQ ID NO: 166 - protein, 12 aa, Artificial Sequence
  Description of Artificial Sequence Synthetic peptide
KFKFFFRRNPYF

SEQ ID NO: 167 - protein, 12 aa, Artificial Sequence
  Description of Artificial Sequence Synthetic peptide
KFLREFWCRHFF

SEQ ID NO: 168 - protein, 12 aa, Artificial Sequence
  Description of Artificial Sequence Synthetic peptide
KFLRFRRSLLLL

SEQ ID NO: 169 - protein, 12 aa, Artificial Sequence
  Description of Artificial Sequence Synthetic peptide
KFRFFYPIRRIA

SEQ ID NO: 170 - protein, 12 aa, Artificial Sequence
  Description of Artificial Sequence Synthetic peptide
KFRLFYPLRRIA

SEQ ID NO: 171 - protein, 12 aa, Artificial Sequence
  Description of Artificial Sequence Synthetic peptide
KFRTWRQLWLWL

SEQ ID NO: 172 - protein, 12 aa, Artificial Sequence
  Description of Artificial Sequence Synthetic peptide
KFRYVWCWPMWR

SEQ ID NO: 173 - protein, 12 aa, Artificial Sequence
  Description of Artificial Sequence Synthetic peptide
KFSRLRRFLWFR

SEQ ID NO: 174 - protein, 12 aa, Artificial Sequence
  Description of Artificial Sequence Synthetic peptide
KIPLFMIKRKIW

SEQ ID NO: 175 - protein, 12 aa, Artificial Sequence
  Description of Artificial Sequence Synthetic peptide
KKFFYCFWWGLR

SEQ ID NO: 176 - protein, 12 aa, Artificial Sequence
  Description of Artificial Sequence Synthetic peptide
KLFFLVHYFVRR

SEQ ID NO: 177 - protein, 12 aa, Artificial Sequence
  Description of Artificial Sequence Synthetic peptide
KLRWVRPLRRIL

SEQ ID NO: 178 - protein, 12 aa, Artificial Sequence
  Description of Artificial Sequence Synthetic peptide
KLWLYKFIRRKF

SEQ ID NO: 179 - protein, 12 aa, Artificial Sequence
  Description of Artificial Sequence Synthetic peptide
KLYYFIRKIKMW

SEQ ID NO: 180 - protein, 12 aa, Artificial Sequence
  Description of Artificial Sequence Synthetic peptide
KMWFVFRMYGIK

SEQ ID NO: 181 - protein, 12 aa, Artificial Sequence
  Description of Artificial Sequence Synthetic peptide
KNFWRRYIYLWI

SEQ ID NO: 182 - protein, 12 aa, Artificial Sequence
  Description of Artificial Sequence Synthetic peptide
KRFAILRKWFCL

SEQ ID NO: 183 - protein, 12 aa, Artificial Sequence
  Description of Artificial Sequence Synthetic peptide
KRFLLLFSFLKR

SEQ ID NO: 184 - protein, 12 aa, Artificial Sequence
  Description of Artificial Sequence Synthetic peptide
KRHWLRFFYLYH

SEQ ID NO: 185 - protein, 12 aa, Artificial Sequence
  Description of Artificial Sequence Synthetic peptide
KRIFLLIFFKRR

SEQ ID NO: 186 - protein, 12 aa, Artificial Sequence
  Description of Artificial Sequence Synthetic peptide
KRLRLLRRWYRP

SEQ ID NO: 187 - protein, 12 aa, Artificial Sequence
  Description of Artificial Sequence Synthetic peptide
KRPVFIFEWLRF

SEQ ID NO: 188 - protein, 12 aa, Artificial Sequence
  Description of Artificial Sequence Synthetic peptide
KRPVFIFEWLRF

SEQ ID NO: 189 - protein, 12 aa, Artificial Sequence
  Description of Artificial Sequence Synthetic peptide
KRRFYRLIMFRC

SEQ ID NO: 190 - protein, 12 aa, Artificial Sequence
  Description of Artificial Sequence Synthetic peptide
KRSWWLLLLKRW

SEQ ID NO: 191 - protein, 12 aa, Artificial Sequence
  Description of Artificial Sequence Synthetic peptide
KRSWWLLLLKRW

SEQ ID NO: 192 - protein, 12 aa, Artificial Sequence
  Description of Artificial Sequence Synthetic peptide
KRSWWLLLLKRW

SEQ ID NO: 193 - protein, 12 aa, Artificial Sequence
  Description of Artificial Sequence Synthetic peptide
KRSWWLLLLKRW

SEQ ID NO: 194 - protein, 12 aa, Artificial Sequence
  Description of Artificial Sequence Synthetic peptide
KRSWWWLLLKRW

SEQ ID NO: 195 - protein, 12 aa, Artificial Sequence
  Description of Artificial Sequence Synthetic peptide
KWWLCFARRRFM

SEQ ID NO: 196 - protein, 12 aa, Artificial Sequence
  Description of Artificial Sequence Synthetic peptide
LAILKRRWWKYM

SEQ ID NO: 197 - protein, 12 aa, Artificial Sequence
  Description of Artificial Sequence Synthetic peptide
LARLLLYRRKLW

SEQ ID NO: 198 - protein, 12 aa, Artificial Sequence
  Description of Artificial Sequence Synthetic peptide
LARRRWHWPWWA

SEQ ID NO: 199 - protein, 12 aa, Artificial Sequence
  Description of Artificial Sequence Synthetic peptide
LFCWAWLPRRRR

SEQ ID NO: 200 - protein, 12 aa, Artificial Sequence
  Description of Artificial Sequence Synthetic peptide
LFFKVFWRKFLR

SEQ ID NO: 201 - protein, 12 aa, Artificial Sequence
  Description of Artificial Sequence Synthetic peptide
LFFRYRARNFFI

SEQ ID NO: 202 - protein, 12 aa, Artificial Sequence
  Description of Artificial Sequence Synthetic peptide
LFILKIFIRRIN

SEQ ID NO: 203 - protein, 12 aa, Artificial Sequence
  Description of Artificial Sequence Synthetic peptide
LFIRRPILWMKK

SEQ ID NO: 204 - protein, 12 aa, Artificial Sequence
  Description of Artificial Sequence Synthetic peptide
LFLLGAIRIWRR

SEQ ID NO: 205 - protein, 12 aa, Artificial Sequence
  Description of Artificial Sequence Synthetic peptide
LFLRIPFIRNKF

SEQ ID NO: 206 - protein, 12 aa, Artificial Sequence
  Description of Artificial Sequence Synthetic peptide
LFLRYRAMFRLR

SEQ ID NO: 207 - protein, 12 aa, Artificial Sequence
  Description of Artificial Sequence Synthetic peptide
LFQRRMLFWHRF

SEQ ID NO: 208 - protein, 12 aa, Artificial Sequence
  Description of Artificial Sequence Synthetic peptide
LFQRRMLYWHRF

SEQ ID NO: 209 - protein, 12 aa, Artificial Sequence
  Description of Artificial Sequence Synthetic peptide
LFRKFRRFDFLF

SEQ ID NO: 210 - protein, 12 aa, Artificial Sequence
  Description of Artificial Sequence Synthetic peptide
LFVVFFFRNPRR

SEQ ID NO: 211 - protein, 12 aa, Artificial Sequence
  Description of Artificial Sequence Synthetic peptide
LGFLFYWRHRYR

SEQ ID NO: 212 - protein, 12 aa, Artificial Sequence
  Description of Artificial Sequence Synthetic peptide
LGIFRRCWLVFK

SEQ ID NO: 213 - protein, 12 aa, Artificial Sequence
  Description of Artificial Sequence Synthetic peptide
LILFWKFVRPKY

SEQ ID NO: 214 - protein, 12 aa, Artificial Sequence
  Description of Artificial Sequence Synthetic peptide
LILKKKMYIFYF

SEQ ID NO: 215 - protein, 12 aa, Artificial Sequence
  Description of Artificial Sequence Synthetic peptide
LIRFMLKLLIKK

SEQ ID NO: 216 - protein, 12 aa, Artificial Sequence
  Description of Artificial Sequence Synthetic peptide
LIVRPFVFRKYL

SEQ ID NO: 217 - protein, 12 aa, Artificial Sequence
  Description of Artificial Sequence Synthetic peptide
LKAFFIRRYFVF

SEQ ID NO: 218 - protein, 12 aa, Artificial Sequence
  Description of Artificial Sequence Synthetic peptide
LKAFFIRRYFVF

SEQ ID NO: 219 - protein, 12 aa, Artificial Sequence
  Description of Artificial Sequence Synthetic peptide
LKIFRRPRKLFM

SEQ ID NO: 220 - protein, 12 aa, Artificial Sequence
  Description of Artificial Sequence Synthetic peptide
LKKFYRGRIWYF

SEQ ID NO: 221 - protein, 12 aa, Artificial Sequence
  Description of Artificial Sequence Synthetic peptide
LKRYAWKRRWFV

SEQ ID NO: 222 - protein, 12 aa, Artificial Sequence
  Description of Artificial Sequence Synthetic peptide
LKRYAWKRRWFV

SEQ ID NO: 223 - protein, 12 aa, Artificial Sequence
  Description of Artificial Sequence Synthetic peptide
LLAILRRRWWKY

SEQ ID NO: 224 - protein, 12 aa, Artificial Sequence
  Description of Artificial Sequence Synthetic peptide
LLFFFVMYKKRL

SEQ ID NO: 225 - protein, 12 aa, Artificial Sequence
  Description of Artificial Sequence Synthetic peptide
LLIIFPWRRRSW

SEQ ID NO: 226 - protein, 12 aa, Artificial Sequence
  Description of Artificial Sequence Synthetic peptide
LLILLKYRWRKL

SEQ ID NO: 227 - protein, 12 aa, Artificial Sequence
  Description of Artificial Sequence Synthetic peptide
LLKICRFFNRFW

SEQ ID NO: 228 - protein, 12 aa, Artificial Sequence
  Description of Artificial Sequence Synthetic peptide
LLMLIFLRQRIR

SEQ ID NO: 229 - protein, 12 aa, Artificial Sequence
  Description of Artificial Sequence Synthetic peptide
LLPLLYYFLKKR

SEQ ID NO: 230 - protein, 12 aa, Artificial Sequence
  Description of Artificial Sequence Synthetic peptide
LLPLRWLPLRRL

SEQ ID NO: 231 - protein, 12 aa, Artificial Sequence
  Description of Artificial Sequence Synthetic peptide
LLQRRMLFWHRF

SEQ ID NO: 232 - protein, 12 aa, Artificial Sequence
  Description of Artificial Sequence Synthetic peptide
LLQRRMLFWHRF

SEQ ID NO: 233 - protein, 12 aa, Artificial Sequence
  Description of Artificial Sequence Synthetic peptide
LLRFLLRKLYVF

SEQ ID NO: 234 - protein, 12 aa, Artificial Sequence
  Description of Artificial Sequence Synthetic peptide
LLRIVFRKRKIF

SEQ ID NO: 235 - protein, 12 aa, Artificial Sequence
  Description of Artificial Sequence Synthetic peptide
LLVVVRLWLRRY

SEQ ID NO: 236 - protein, 12 aa, Artificial Sequence
  Description of Artificial Sequence Synthetic peptide
LLWMPKRLLKYI

SEQ ID NO: 237 - protein, 12 aa, Artificial Sequence
  Description of Artificial Sequence Synthetic peptide
LMIILWKYLRKP

SEQ ID NO: 238 - protein, 12 aa, Artificial Sequence
  Description of Artificial Sequence Synthetic peptide
LMKFFPFEKRYF

SEQ ID NO: 239 - protein, 12 aa, Artificial Sequence
  Description of Artificial Sequence Synthetic peptide
LMPWRWLPRKPL

SEQ ID NO: 240 - protein, 12 aa, Artificial Sequence
  Description of Artificial Sequence Synthetic peptide
LMRIFRILKLAR

SEQ ID NO: 241 - protein, 12 aa, Artificial Sequence
  Description of Artificial Sequence Synthetic peptide
LPFPLRRLLWRC

SEQ ID NO: 242 - protein, 12 aa, Artificial Sequence
  Description of Artificial Sequence Synthetic peptide
LPRLFRFLQRPL

SEQ ID NO: 243 - protein, 12 aa, Artificial Sequence
  Description of Artificial Sequence Synthetic peptide
LRFLFWKVYKRL

SEQ ID NO: 244 - protein, 12 aa, Artificial Sequence
  Description of Artificial Sequence Synthetic peptide
LRILPKILFMRR

SEQ ID NO: 245 - protein, 12 aa, Artificial Sequence
  Description of Artificial Sequence Synthetic peptide
LRPAMRLRLRFI

SEQ ID NO: 246 - protein, 12 aa, Artificial Sequence
  Description of Artificial Sequence Synthetic peptide
LRRFLRFDFYMR

SEQ ID NO: 247 - protein, 12 aa, Artificial Sequence
  Description of Artificial Sequence Synthetic peptide
LRRFYRGRIWYL

SEQ ID NO: 248 - protein, 12 aa, Artificial Sequence
  Description of Artificial Sequence Synthetic peptide
LRRFYRGRIWYL

SEQ ID NO: 249 - protein, 12 aa, Artificial Sequence
  Description of Artificial Sequence Synthetic peptide
LRRIILLQRWFR

SEQ ID NO: 250 - protein, 12 aa, Artificial Sequence
  Description of Artificial Sequence Synthetic peptide
LRRIVLLQRWFR

SEQ ID NO: 251 - protein, 12 aa, Artificial Sequence
  Description of Artificial Sequence Synthetic peptide
LSFWGFKKIRWF

SEQ ID NO: 252 - protein, 12 aa, Artificial Sequence
  Description of Artificial Sequence Synthetic peptide
LVILKRKWWKYI

SEQ ID NO: 253 - protein, 12 aa, Artificial Sequence
  Description of Artificial Sequence Synthetic peptide
LWAFERIKRFVF

SEQ ID NO: 254 - protein, 12 aa, Artificial Sequence
  Description of Artificial Sequence Synthetic peptide
LWFHFKRYRYRR

SEQ ID NO: 255 - protein, 12 aa, Artificial Sequence
  Description of Artificial Sequence Synthetic peptide
LWKMGFFRRRYK

SEQ ID NO: 256 - protein, 12 aa, Artificial Sequence
  Description of Artificial Sequence Synthetic peptide
LWLLFVPPRVRR

SEQ ID NO: 257 - protein, 12 aa, Artificial Sequence
  Description of Artificial Sequence Synthetic peptide
LWWLRFRRPHPI

SEQ ID NO: 258 - protein, 12 aa, Artificial Sequence
  Description of Artificial Sequence Synthetic peptide
LWYFRKRWCALV

SEQ ID NO: 259 - protein, 12 aa, Artificial Sequence
  Description of Artificial Sequence Synthetic peptide
LYFFHKKILRIL

SEQ ID NO: 260 - protein, 12 aa, Artificial Sequence
  Description of Artificial Sequence Synthetic peptide
LYFRIRFYFRNW

SEQ ID NO: 261 - protein, 12 aa, Artificial Sequence
  Description of Artificial Sequence Synthetic peptide
LYLIYRKFFFKK

SEQ ID NO: 262 - protein, 12 aa, Artificial Sequence
  Description of Artificial Sequence Synthetic peptide
LYQRRMLFWHRF

SEQ ID NO: 263 - protein, 12 aa, Artificial Sequence
  Description of Artificial Sequence Synthetic peptide
LYRFFKRIHLGW

SEQ ID NO: 264 - protein, 12 aa, Artificial Sequence
  Description of Artificial Sequence Synthetic peptide
LYYLLRAMRRFV

SEQ ID NO: 265 - protein, 12 aa, Artificial Sequence
  Description of Artificial Sequence Synthetic peptide
MAAMRWRWWQRL

SEQ ID NO: 266 - protein, 12 aa, Artificial Sequence
  Description of Artificial Sequence Synthetic peptide
MAFRWRSLMRFR

SEQ ID NO: 267 - protein, 12 aa, Artificial Sequence
  Description of Artificial Sequence Synthetic peptide
MAKLWFKFQRYF

SEQ ID NO: 268 - protein, 12 aa, Artificial Sequence
  Description of Artificial Sequence Synthetic peptide
MALFRKFFFKKP

SEQ ID NO: 269 - protein, 12 aa, Artificial Sequence
  Description of Artificial Sequence Synthetic peptide
MALFRKFFYRKP

SEQ ID NO: 270 - protein, 12 aa, Artificial Sequence
  Description of Artificial Sequence Synthetic peptide
MARFFRRRKFCR

SEQ ID NO: 271 - protein, 12 aa, Artificial Sequence
  Description of Artificial Sequence Synthetic peptide
MARFFRRRKFCR

SEQ ID NO: 272 - protein, 12 aa, Artificial Sequence
  Description of Artificial Sequence Synthetic peptide
MARFFRRRKFCR

SEQ ID NO: 273 - protein, 12 aa, Artificial Sequence
  Description of Artificial Sequence Synthetic peptide
MAWGWWKRKRRW

SEQ ID NO: 274 - protein, 12 aa, Artificial Sequence
  Description of Artificial Sequence Synthetic peptide
MAWGWWRRWRRW

SEQ ID NO: 275 - protein, 12 aa, Artificial Sequence
  Description of Artificial Sequence Synthetic peptide
MAWPWRRRRWRW

SEQ ID NO: 276 - protein, 12 aa, Artificial Sequence
  Description of Artificial Sequence Synthetic peptide
MAWWWGRWRRRW

SEQ ID NO: 277 - protein, 12 aa, Artificial Sequence
  Description of Artificial Sequence Synthetic peptide
MAWYWWRRRRRR

SEQ ID NO: 278 - protein, 12 aa, Artificial Sequence
  Description of Artificial Sequence Synthetic peptide
MAWYWWRRRRRR

SEQ ID NO: 279 - protein, 12 aa, Artificial Sequence
  Description of Artificial Sequence Synthetic peptide
MAWYWWRRRRRR

SEQ ID NO: 280 - protein, 12 aa, Artificial Sequence
  Description of Artificial Sequence Synthetic peptide
MFFFFRFRSKRW

SEQ ID NO: 281 - protein, 12 aa, Artificial Sequence
  Description of Artificial Sequence Synthetic peptide
MFFFWKKVKRIH

SEQ ID NO: 282 - protein, 12 aa, Artificial Sequence
  Description of Artificial Sequence Synthetic peptide
MFFKWISKFIRR

SEQ ID NO: 283 - protein, 12 aa, Artificial Sequence
  Description of Artificial Sequence Synthetic peptide
MFFNFKKYFLIK

SEQ ID NO: 284 - protein, 12 aa, Artificial Sequence
  Description of Artificial Sequence Synthetic peptide
MFIFRGRKFWAL

SEQ ID NO: 285 - protein, 12 aa, Artificial Sequence
  Description of Artificial Sequence Synthetic peptide
MFYLIKKLPKFI

SEQ ID NO: 286 - protein, 12 aa, Artificial Sequence
  Description of Artificial Sequence Synthetic peptide
MIRIRNRWFRWL

SEQ ID NO: 287 - protein, 12 aa, Artificial Sequence
  Description of Artificial Sequence Synthetic peptide
MIYRRFKFRNFI

SEQ ID NO: 288 - protein, 12 aa, Artificial Sequence
  Description of Artificial Sequence Synthetic peptide
MIYRYLRPWLFK

SEQ ID NO: 289 - protein, 12 aa, Artificial Sequence
  Description of Artificial Sequence Synthetic peptide
MKIWRFFLMKER

SEQ ID NO: 290 - protein, 12 aa, Artificial Sequence
  Description of Artificial Sequence Synthetic peptide
MKIYFWKLKFFF

SEQ ID NO: 291 - protein, 12 aa, Artificial Sequence
  Description of Artificial Sequence Synthetic peptide
MKKWRYYFVNFW

SEQ ID NO: 292 - protein, 12 aa, Artificial Sequence
  Description of Artificial Sequence Synthetic peptide
MKLFWVKRLLRI

SEQ ID NO: 293 - protein, 12 aa, Artificial Sequence
  Description of Artificial Sequence Synthetic peptide
MKLLAFRRLLRI

SEQ ID NO: 294 - protein, 12 aa, Artificial Sequence
  Description of Artificial Sequence Synthetic peptide
MKMILVRRFRVL

SEQ ID NO: 295 - protein, 12 aa, Artificial Sequence
  Description of Artificial Sequence Synthetic peptide
MKRRRRWRGWLL

SEQ ID NO: 296 - protein, 12 aa, Artificial Sequence
  Description of Artificial Sequence Synthetic peptide
MKWLFKYLIRFY

SEQ ID NO: 297 - protein, 12 aa, Artificial Sequence
  Description of Artificial Sequence Synthetic peptide
MKYLLIKFVRFW

SEQ ID NO: 298 - protein, 12 aa, Artificial Sequence
  Description of Artificial Sequence Synthetic peptide
MLFYRFKSWYRL

SEQ ID NO: 299 - protein, 12 aa, Artificial Sequence
  Description of Artificial Sequence Synthetic peptide
MLIWWRGKFRRA

SEQ ID NO: 300 - protein, 12 aa, Artificial Sequence
  Description of Artificial Sequence Synthetic peptide
MLKFFLKLRKRR

SEQ ID NO: 301 - protein, 12 aa, Artificial Sequence
  Description of Artificial Sequence Synthetic peptide
MLKFLLKFRKRR

SEQ ID NO: 302 - protein, 12 aa, Artificial Sequence
  Description of Artificial Sequence Synthetic peptide
MLLKIKIKIRLF

SEQ ID NO: 303 - protein, 12 aa, Artificial Sequence
  Description of Artificial Sequence Synthetic peptide
MLLLRWKRFWFL

SEQ ID NO: 304 - protein, 12 aa, Artificial Sequence
  Description of Artificial Sequence Synthetic peptide
MLVLRKFRWRKW

SEQ ID NO: 305 - protein, 12 aa, Artificial Sequence
  Description of Artificial Sequence Synthetic peptide
MLWPFRWVWWKR

SEQ ID NO: 306 - protein, 12 aa, Artificial Sequence
  Description of Artificial Sequence Synthetic peptide
MMFWRIFRLELR

SEQ ID NO: 307 - protein, 12 aa, Artificial Sequence
  Description of Artificial Sequence Synthetic peptide
MMKMARFFYRLP

SEQ ID NO: 308 - protein, 12 aa, Artificial Sequence
  Description of Artificial Sequence Synthetic peptide
MMPRLLFRAWPR

SEQ ID NO: 309 - protein, 12 aa, Artificial Sequence
  Description of Artificial Sequence Synthetic peptide
MPRIFPWKLWRK

SEQ ID NO: 310 - protein, 12 aa, Artificial Sequence
  Description of Artificial Sequence Synthetic peptide
MPWWPWRRWRRW

SEQ ID NO: 311 - protein, 12 aa, Artificial Sequence
  Description of Artificial Sequence Synthetic peptide
MRFFKKYLYYRI

SEQ ID NO: 312 - protein, 12 aa, Artificial Sequence
  Description of Artificial Sequence Synthetic peptide
MRFLRWFHKWRQ

SEQ ID NO: 313 - protein, 12 aa, Artificial Sequence
  Description of Artificial Sequence Synthetic peptide
MRFLRWIRQIWR

SEQ ID NO: 314 - protein, 12 aa, Artificial Sequence
  Description of Artificial Sequence Synthetic peptide
MRFLSFRRLLLY

SEQ ID NO: 315 - protein, 12 aa, Artificial Sequence
  Description of Artificial Sequence Synthetic peptide
MRFVFFMMKHKW

SEQ ID NO: 316 - protein, 12 aa, Artificial Sequence
  Description of Artificial Sequence Synthetic peptide
MRIFRPWRLRCP

SEQ ID NO: 317 - protein, 12 aa, Artificial Sequence
  Description of Artificial Sequence Synthetic peptide
MRKWLYRLFIEL

SEQ ID NO: 318 - protein, 12 aa, Artificial Sequence
  Description of Artificial Sequence Synthetic peptide
MRNRWIWRFLRP

SEQ ID NO: 319 - protein, 12 aa, Artificial Sequence
  Description of Artificial Sequence Synthetic peptide
MRSRWIWRFLRP

SEQ ID NO: 320 - protein, 12 aa, Artificial Sequence
  Description of Artificial Sequence Synthetic peptide
MRTLLIRYILWR

SEQ ID NO: 321 - protein, 12 aa, Artificial Sequence
  Description of Artificial Sequence Synthetic peptide
MRTLLIRYILWR

SEQ ID NO: 322 - protein, 12 aa, Artificial Sequence
  Description of Artificial Sequence Synthetic peptide
MRYFYVKWPFFK

SEQ ID NO: 323 - protein, 12 aa, Artificial Sequence
  Description of Artificial Sequence Synthetic peptide
MSRFWHFKKFYF

SEQ ID NO: 324 - protein, 12 aa, Artificial Sequence
  Description of Artificial Sequence Synthetic peptide
MVFCLILWKWKK

SEQ ID NO: 325 - protein, 12 aa, Artificial Sequence
  Description of Artificial Sequence Synthetic peptide
MVLKFFRWLFRL

SEQ ID NO: 326 - protein, 12 aa, Artificial Sequence
  Description of Artificial Sequence Synthetic peptide
MVLRRLLRKRWV

SEQ ID NO: 327 - protein, 12 aa, Artificial Sequence
  Description of Artificial Sequence Synthetic peptide
MVRILRWFIRLY

SEQ ID NO: 328 - protein, 12 aa, Artificial Sequence
  Description of Artificial Sequence Synthetic peptide
MVRILRWFIRLY

SEQ ID NO: 329 - protein, 12 aa, Artificial Sequence
  Description of Artificial Sequence Synthetic peptide
MVRILRWFIRLY

SEQ ID NO: 330 - protein, 12 aa, Artificial Sequence
  Description of Artificial Sequence Synthetic peptide
MVRILRWFIRLY

SEQ ID NO: 331 - protein, 12 aa, Artificial Sequence
  Description of Artificial Sequence Synthetic peptide
MVRILRWFIRLY

SEQ ID NO: 332 - protein, 12 aa, Artificial Sequence
  Description of Artificial Sequence Synthetic peptide
MVRILRWFIRLY

SEQ ID NO: 333 - protein, 12 aa, Artificial Sequence
  Description of Artificial Sequence Synthetic peptide
MVWFKRVKPFIR

SEQ ID NO: 334 - protein, 12 aa, Artificial Sequence
  Description of Artificial Sequence Synthetic peptide
MWCIRLRYLRLL

SEQ ID NO: 335 - protein, 12 aa, Artificial Sequence
  Description of Artificial Sequence Synthetic peptide
MWFRNLIPYRLR

SEQ ID NO: 336 - protein, 12 aa, Artificial Sequence
  Description of Artificial Sequence Synthetic peptide
MWKLWKFVDFRM

SEQ ID NO: 337 - protein, 12 aa, Artificial Sequence
  Description of Artificial Sequence Synthetic peptide
MWRIRRRYCRLL

SEQ ID NO: 338 - protein, 12 aa, Artificial Sequence
  Description of Artificial Sequence Synthetic peptide
MWRIWRLFDPMR

SEQ ID NO: 339 - protein, 12 aa, Artificial Sequence
  Description of Artificial Sequence Synthetic peptide
MWWWRRRFWRPK

SEQ ID NO: 340 - protein, 12 aa, Artificial Sequence
  Description of Artificial Sequence Synthetic peptide
MYFKKRRWFLIL

SEQ ID NO: 341 - protein, 12 aa, Artificial Sequence
  Description of Artificial Sequence Synthetic peptide
MYKIFFRLVFKR

SEQ ID NO: 342 - protein, 12 aa, Artificial Sequence
  Description of Artificial Sequence Synthetic peptide
MYKLFFRLVFKR

SEQ ID NO: 343 - protein, 12 aa, Artificial Sequence
  Description of Artificial Sequence Synthetic peptide
NILRILFWFGRR

SEQ ID NO: 344 - protein, 12 aa, Artificial Sequence
  Description of Artificial Sequence Synthetic peptide
NLWKFWLRRRVY

SEQ ID NO: 345 - protein, 12 aa, Artificial Sequence
  Description of Artificial Sequence Synthetic peptide
PFMRWRDRFLFC

SEQ ID NO: 346 - protein, 12 aa, Artificial Sequence
  Description of Artificial Sequence Synthetic peptide
PFMRWRDRFLFC

SEQ ID NO: 347 - protein, 12 aa, Artificial Sequence
  Description of Artificial Sequence Synthetic peptide
PFMRWRDRFLFC

SEQ ID NO: 348 - protein, 12 aa, Artificial Sequence
  Description of Artificial Sequence Synthetic peptide
PFMRWRDRFLFC

SEQ ID NO: 349 - protein, 12 aa, Artificial Sequence
  Description of Artificial Sequence Synthetic peptide
PFMRWRDRFLFC

SEQ ID NO: 350 - protein, 12 aa, Artificial Sequence
  Description of Artificial Sequence Synthetic peptide
PFMRWRDRFLFC

SEQ ID NO: 351 - protein, 12 aa, Artificial Sequence
  Description of Artificial Sequence Synthetic peptide
PFMRWRDRFLFC

SEQ ID NO: 352 - protein, 12 aa, Artificial Sequence
  Description of Artificial Sequence Synthetic peptide
PFMRWRDRFLFC

SEQ ID NO: 353 - protein, 12 aa, Artificial Sequence
  Description of Artificial Sequence Synthetic peptide
PFMRWRDRFLFC

SEQ ID NO: 354 - protein, 12 aa, Artificial Sequence
  Description of Artificial Sequence Synthetic peptide
PFMRWRDRFLFC

SEQ ID NO: 355 - protein, 12 aa, Artificial Sequence
  Description of Artificial Sequence Synthetic peptide
PFMRWRDRFLFC

SEQ ID NO: 356 - protein, 12 aa, Artificial Sequence
  Description of Artificial Sequence Synthetic peptide
PFMRWRDRFLFC

SEQ ID NO: 357 - protein, 12 aa, Artificial Sequence
  Description of Artificial Sequence Synthetic peptide
PFMRWRDRFLFC

SEQ ID NO: 358 - protein, 12 aa, Artificial Sequence
  Description of Artificial Sequence Synthetic peptide
PFMRWRDRFLFC

SEQ ID NO: 359 - protein, 12 aa, Artificial Sequence
  Description of Artificial Sequence Synthetic peptide
PFMRWRDRFLFC

SEQ ID NO: 360 - protein, 12 aa, Artificial Sequence
  Description of Artificial Sequence Synthetic peptide
PFMRWRDRFLFR

SEQ ID NO: 361 - protein, 12 aa, Artificial Sequence
  Description of Artificial Sequence Synthetic peptide
PFMRWRDRFLFV

SEQ ID NO: 362 - protein, 12 aa, Artificial Sequence
  Description of Artificial Sequence Synthetic peptide
PFMRWRDRFLFV

SEQ ID NO: 363 - protein, 12 aa, Artificial Sequence
  Description of Artificial Sequence Synthetic peptide
PFRPWYFAMRLK

SEQ ID NO: 364 - protein, 12 aa, Artificial Sequence
  Description of Artificial Sequence Synthetic peptide
PIFIRRLHRLLL

SEQ ID NO: 365 - protein, 12 aa, Artificial Sequence
  Description of Artificial Sequence Synthetic peptide
PIFIRRLHRLLL

SEQ ID NO: 366 - protein, 12 aa, Artificial Sequence
  Description of Artificial Sequence Synthetic peptide
PLFIPYLRKLKF

SEQ ID NO: 367 - protein, 12 aa, Artificial Sequence
  Description of Artificial Sequence Synthetic peptide
PLLAYRRFWWKK

SEQ ID NO: 368 - protein, 12 aa, Artificial Sequence
  Description of Artificial Sequence Synthetic peptide
PLRKLKVYFIFY

SEQ ID NO: 369 - protein, 12 aa, Artificial Sequence
  Description of Artificial Sequence Synthetic peptide
PLWRLYRGRVWY

SEQ ID NO: 370 - protein, 12 aa, Artificial Sequence
  Description of Artificial Sequence Synthetic peptide
QLKFRLFYFLRR

SEQ ID NO: 371 - protein, 12 aa, Artificial Sequence
  Description of Artificial Sequence Synthetic peptide
RALLRWFRRSFF

SEQ ID NO: 372 - protein, 12 aa, Artificial Sequence
  Description of Artificial Sequence Synthetic peptide
RFFIPYLRKLKF

SEQ ID NO: 373 - protein, 12 aa, Artificial Sequence
  Description of Artificial Sequence Synthetic peptide
RFKLFRMWLAKL

SEQ ID NO: 374 - protein, 12 aa, Artificial Sequence
  Description of Artificial Sequence Synthetic peptide
RFKLLRMWLAKL

SEQ ID NO: 375 - protein, 12 aa, Artificial Sequence
  Description of Artificial Sequence Synthetic peptide
RFLWKRWYLNKL

SEQ ID NO: 376 - protein, 12 aa, Artificial Sequence
  Description of Artificial Sequence Synthetic peptide
RFLWLTLFKIRK

SEQ ID NO: 377 - protein, 12 aa, Artificial Sequence
  Description of Artificial Sequence Synthetic peptide
RFRLPFRRPPIR

SEQ ID NO: 378 - protein, 12 aa, Artificial Sequence
  Description of Artificial Sequence Synthetic peptide
RFRWRRRLFVIS

SEQ ID NO: 379 - protein, 12 aa, Artificial Sequence
  Description of Artificial Sequence Synthetic peptide
RFYIRLIRKRAW

SEQ ID NO: 380 - protein, 12 aa, Artificial Sequence
  Description of Artificial Sequence Synthetic peptide
RFYMLLYVFLKR

SEQ ID NO: 381 - protein, 12 aa, Artificial Sequence
  Description of Artificial Sequence Synthetic peptide
RGFKRLYFRFFK

SEQ ID NO: 382 - protein, 12 aa, Artificial Sequence
  Description of Artificial Sequence Synthetic peptide
RGFRVLYWRLGW

SEQ ID NO: 383 - protein, 12 aa, Artificial Sequence
  Description of Artificial Sequence Synthetic peptide
RIFIVQKIFWIK

SEQ ID NO: 384 - protein, 12 aa, Artificial Sequence
  Description of Artificial Sequence Synthetic peptide
RIFWYRHFRYFI

SEQ ID NO: 385 - protein, 12 aa, Artificial Sequence
  Description of Artificial Sequence Synthetic peptide
RILRLFRRRMMF

SEQ ID NO: 386 - protein, 12 aa, Artificial Sequence
  Description of Artificial Sequence Synthetic peptide
RLFRRFRPRARF

SEQ ID NO: 387 - protein, 12 aa, Artificial Sequence
  Description of Artificial Sequence Synthetic peptide
RLIRKFYYFLKY

SEQ ID NO: 388 - protein, 12 aa, Artificial Sequence
  Description of Artificial Sequence Synthetic peptide
RLKMLVFRLIRR

SEQ ID NO: 389 - protein, 12 aa, Artificial Sequence
  Description of Artificial Sequence Synthetic peptide
RLRLLFWVARFQ

SEQ ID NO: 390 - protein, 12 aa, Artificial Sequence
  Description of Artificial Sequence Synthetic peptide
RPRIAVRRWLFL

SEQ ID NO: 391 - protein, 12 aa, Artificial Sequence
  Description of Artificial Sequence Synthetic peptide
RQLFRFYFKYIM

SEQ ID NO: 392 - protein, 12 aa, Artificial Sequence
  Description of Artificial Sequence Synthetic peptide
RRIILLQRWFRV

SEQ ID NO: 393 - protein, 12 aa, Artificial Sequence
  Description of Artificial Sequence Synthetic peptide
RRIWWRFHLYSI

SEQ ID NO: 394 - protein, 12 aa, Artificial Sequence
  Description of Artificial Sequence Synthetic peptide
RRKMMPRWWGWL

SEQ ID NO: 395 - protein, 12 aa, Artificial Sequence
  Description of Artificial Sequence Synthetic peptide
RRWCPPPFFYRR

SEQ ID NO: 396 - protein, 12 aa, Artificial Sequence
  Description of Artificial Sequence Synthetic peptide
RVYLLRLRFRLV

SEQ ID NO: 397 - protein, 12 aa, Artificial Sequence
  Description of Artificial Sequence Synthetic peptide
RWLLLQLIKFVR

SEQ ID NO: 398 - protein, 12 aa, Artificial Sequence
  Description of Artificial Sequence Synthetic peptide
RWMYLRRFIIYL

SEQ ID NO: 399 - protein, 12 aa, Artificial Sequence
  Description of Artificial Sequence Synthetic peptide
RYRIPREILFWL

SEQ ID NO: 400 - protein, 12 aa, Artificial Sequence
  Description of Artificial Sequence Synthetic peptide
SFFRAFFRVPKW

SEQ ID NO: 401 - protein, 12 aa, Artificial Sequence
  Description of Artificial Sequence Synthetic peptide
SWKFRLFYLLRR

SEQ ID NO: 402 - protein, 12 aa, Artificial Sequence
  Description of Artificial Sequence Synthetic peptide
TFFFAMMLRKFK

SEQ ID NO: 403 - protein, 12 aa, Artificial Sequence
  Description of Artificial Sequence Synthetic peptide
TLIFFRKILWKI

SEQ ID NO: 404 - protein, 12 aa, Artificial Sequence
  Description of Artificial Sequence Synthetic peptide
VFIRLFRRLQWG

SEQ ID NO: 405 - protein, 12 aa, Artificial Sequence
  Description of Artificial Sequence Synthetic peptide
VFKNLYFFYFRR

SEQ ID NO: 406 - protein, 12 aa, Artificial Sequence
  Description of Artificial Sequence Synthetic peptide
VFKQLYFFYFKR

SEQ ID NO: 407 - protein, 12 aa, Artificial Sequence
  Description of Artificial Sequence Synthetic peptide
VFRLRFGYFIKR

SEQ ID NO: 408 - protein, 12 aa, Artificial Sequence
  Description of Artificial Sequence Synthetic peptide
VFRRFVWNFFRL

SEQ ID NO: 409 - protein, 12 aa, Artificial Sequence
  Description of Artificial Sequence Synthetic peptide
VFRRFVWNFFRL

SEQ ID NO: 410 - protein, 12 aa, Artificial Sequence
  Description of Artificial Sequence Synthetic peptide
VFRRRRWHYMIL

SEQ ID NO: 411 - protein, 12 aa, Artificial Sequence
  Description of Artificial Sequence Synthetic peptide
VFWVVWRYRRRG

SEQ ID NO: 412 - protein, 12 aa, Artificial Sequence
  Description of Artificial Sequence Synthetic peptide
VIRLVRVFRIFK

SEQ ID NO: 413 - protein, 12 aa, Artificial Sequence
  Description of Artificial Sequence Synthetic peptide
VLFRFRWKYIKH

SEQ ID NO: 414 - protein, 12 aa, Artificial Sequence
  Description of Artificial Sequence Synthetic peptide
VLIKRWPPPLRW

SEQ ID NO: 415 - protein, 12 aa, Artificial Sequence
  Description of Artificial Sequence Synthetic peptide
VLLRVRMLYFLR

SEQ ID NO: 416 - protein, 12 aa, Artificial Sequence
  Description of Artificial Sequence Synthetic peptide
VLPFIYFILRRK

SEQ ID NO: 417 - protein, 12 aa, Artificial Sequence
  Description of Artificial Sequence Synthetic peptide
VRRRRTIILRWW

SEQ ID NO: 418 - protein, 12 aa, Artificial Sequence
  Description of Artificial Sequence Synthetic peptide
VSFGRFLWRRFL

SEQ ID NO: 419 - protein, 12 aa, Artificial Sequence
  Description of Artificial Sequence Synthetic peptide
VSFGRFLWRRFL

SEQ ID NO: 420 - protein, 12 aa, Artificial Sequence
  Description of Artificial Sequence Synthetic peptide
VSFGRFLWRRFL

SEQ ID NO: 421 - protein, 12 aa, Artificial Sequence
  Description of Artificial Sequence Synthetic peptide
VSFGRFLWRRFL

SEQ ID NO: 422 - protein, 12 aa, Artificial Sequence
  Description of Artificial Sequence Synthetic peptide
VVMTRIWKWRLW

SEQ ID NO: 423 - protein, 12 aa, Artificial Sequence
  Description of Artificial Sequence Synthetic peptide
VYFVIRLFRKYM

SEQ ID NO: 424 - protein, 12 aa, Artificial Sequence
  Description of Artificial Sequence Synthetic peptide
VYLFRMRFRLVR

SEQ ID NO: 425 - protein, 12 aa, Artificial Sequence
  Description of Artificial Sequence Synthetic peptide
VYLLRLRFRLVR

SEQ ID NO: 426 - protein, 12 aa, Artificial Sequence
  Description of Artificial Sequence Synthetic peptide
WEYFRLRPLRFR

SEQ ID NO: 427 - protein, 12 aa, Artificial Sequence
  Description of Artificial Sequence Synthetic peptide
WFLYYRFKKRYL

SEQ ID NO: 428 - protein, 12 aa, Artificial Sequence
  Description of Artificial Sequence Synthetic peptide
WFYVFFYRRLKL

SEQ ID NO: 429 - protein, 12 aa, Artificial Sequence
  Description of Artificial Sequence Synthetic peptide
WIPERMLRRYFL

SEQ ID NO: 430 - protein, 12 aa, Artificial Sequence
  Description of Artificial Sequence Synthetic peptide
WIWACIRKRRLI

SEQ ID NO: 431 - protein, 12 aa, Artificial Sequence
  Description of Artificial Sequence Synthetic peptide
WKCFFRRLWARL

SEQ ID NO: 432 - protein, 12 aa, Artificial Sequence
  Description of Artificial Sequence Synthetic peptide
WKFLRLYFYPTR

SEQ ID NO: 433 - protein, 12 aa, Artificial Sequence
  Description of Artificial Sequence Synthetic peptide
WKILWFIPFRQR

SEQ ID NO: 434 - protein, 12 aa, Artificial Sequence
  Description of Artificial Sequence Synthetic peptide
WKILWFIPFRRR

SEQ ID NO: 435 - protein, 12 aa, Artificial Sequence
  Description of Artificial Sequence Synthetic peptide
WLIPYLRRLKFG

SEQ ID NO: 436 - protein, 12 aa, Artificial Sequence
  Description of Artificial Sequence Synthetic peptide
WLIRIILRQMKL

SEQ ID NO: 437 - protein, 12 aa, Artificial Sequence
  Description of Artificial Sequence Synthetic peptide
WLRRFLLYRYLT

SEQ ID NO: 438 - protein, 12 aa, Artificial Sequence
  Description of Artificial Sequence Synthetic peptide
WLYRFFFRFLQK

SEQ ID NO: 439 - protein, 12 aa, Artificial Sequence
  Description of Artificial Sequence Synthetic peptide
WMYKYKTPWFFR

SEQ ID NO: 440 - protein, 12 aa, Artificial Sequence
  Description of Artificial Sequence Synthetic peptide
WRFAIFFLRTMR

SEQ ID NO: 441 - protein, 12 aa, Artificial Sequence
  Description of Artificial Sequence Synthetic peptide
WRRIRWALKLVR

SEQ ID NO: 442 - protein, 12 aa, Artificial Sequence
  Description of Artificial Sequence Synthetic peptide
WWGWRRFLWRRL

SEQ ID NO: 443 - protein, 12 aa, Artificial Sequence
  Description of Artificial Sequence Synthetic peptide
WWLWRTALAWRR

SEQ ID NO: 444 - protein, 12 aa, Artificial Sequence
  Description of Artificial Sequence Synthetic peptide
YFRMRFYFRNWH

SEQ ID NO: 445 - protein, 12 aa, Artificial Sequence
  Description of Artificial Sequence Synthetic peptide
YIFFRYHRYLFK

SEQ ID NO: 446 - protein, 12 aa, Artificial Sequence
  Description of Artificial Sequence Synthetic peptide
YIFIKKKGWFFF

SEQ ID NO: 447 - protein, 12 aa, Artificial Sequence
  Description of Artificial Sequence Synthetic peptide
YKFWLRTYRVFF

SEQ ID NO: 448 - protein, 12 aa, Artificial Sequence
  Description of Artificial Sequence Synthetic peptide
YLALYRRLWFAR

SEQ ID NO: 449 - protein, 12 aa, Artificial Sequence
  Description of Artificial Sequence Synthetic peptide
YMWVRWTIPRFR

SEQ ID NO: 450 - protein, 12 aa, Artificial Sequence
  Description of Artificial Sequence Synthetic peptide
YMWVRWTIPRFR

SEQ ID NO: 451 - protein, 12 aa, Artificial Sequence
  Description of Artificial Sequence Synthetic peptide
YQRMMYWFPRFK

SEQ ID NO: 452 - protein, 12 aa, Artificial Sequence
  Description of Artificial Sequence Synthetic peptide
YVFYLWRRLLKP

SEQ ID NO: 453 - protein, 12 aa, Artificial Sequence
  Description of Artificial Sequence Synthetic peptide
YWPKRARWPRLF

SEQ ID NO: 454 - protein, 12 aa, Artificial Sequence
  Description of Artificial Sequence Synthetic peptide
YWRRFWWLVSPK

SEQ ID NO: 455 - protein, 12 aa, Artificial Sequence
  Description of Artificial Sequence Synthetic peptide
YYIFRRFKTWWA

(19) United States
(12) Patent Application Publication
(10) Pub. No.: US 2018/0094030 A1
(43) Pub. Date: Apr. 5, 2018

(54) CELL PENETRATING PEPTIDES & METHODS OF IDENTIFYING CELL PENETRATING PEPTIDES

(71) Applicant: Hoffmann-La Roche Inc., Nutley, NJ (US)

(72) Inventor: Francesca Milletti, New York, NY (US)

(21) Appl. No.: 15/625,219

(22) Filed: Jun. 16, 2017

Related U.S. Application Data
(63) Continuation of application No. 14/410,930, filed on Dec. 23, 2014, now abandoned.
(60) Provisional application No. 61/664,210, filed on Jun. 26, 2012.

(51) Int. Cl. C07K 7/08; A61K 47/64; C07K 7/06; G01N 33/50

(52) U.S. Cl. CPC C07K 7/08; A61K 47/64; G01N 33/502; C07K 7/06

(57) ABSTRACT

The present invention relates to cell penetrating peptides and methods of identifying cell penetrating peptides based upon hydrophobicity and polarity.

### FIELD OF THE INVENTION

The present invention relates to cell penetrating peptides and methods of identifying cell penetrating peptides based upon hydrophobicity and polarity.

### BACKGROUND OF THE INVENTION

Cell-penetrating peptides (CPPs) such as the antennapedia-derived penetratin (Derossi et al., Biol. Chem., 269, 10444-10450, 1994) and the Tat peptide (Vives et al., J. Biol. Chem., 272, 16010-16017, 1997) are widely used tools for the delivery of cargo molecules such as peptides, proteins and oligonucleotides into cells (Fischer et al., Bioconjug. Chem., 12, 825-841, 2001). Areas of application range from purely cell biological to biomedical research (Dietz and Bahr, Mol. Cell., Neurosci, 27, 85-131, 2004). Initially, cellular uptake was believed to occur by direct permeation of the plasma membrane (Prochiantz, Curr. Opin. Cell Biol., 12, 400-406, 2000). In the past years, evidence has been accumulated that for several CPPs, endocytosis contributes at least significantly to the cellular uptake (for a review, see Fotin-Mleczek et al., Curr. Pharm. Design, 11, 3613-3628, 2005). Given these recent results, the specification of a peptide as a CPP therefore does not imply a specific cellular import mechanism, but rather refers to a function as a peptide that, when conjugated to a cargo, either covalently or non-covalently, enhances the cellular uptake of the cargo molecule.

Most cell penetrating peptides have many hydrophobic and/or positively charged residues, but their vast sequence diversity makes it difficult to predict whether any given peptide will be cell penetrating. Cruciani et al., J. Chemometrics, 2004; 18: 146-155, proposed a set of descriptors (PP1 [polarity] and PP2 [hydrophobicity]) for each of the 20 amino acids. However, despite these descriptors no method was proposed or exists that can reasonably predict the cell penetrating properties of a peptide based upon PP1 and PP2.

### SUMMARY OF THE INVENTION

The present invention relates to cell penetrating peptides and methods of identifying cell penetrating peptides based upon hydrophobicity and polarity.

In one embodiment, the present invention relates to a method of identifying cell penetrating peptides among a group of peptides by: (1) determining the polarity (referred to as the “PP1”) of said peptides; (2) determining the hydrophobicity (referred to as the “PP2”) of said peptides; (3) identifying peptides within the group, wherein PP1<[(PP2*X1)+X], wherein X1 is 1.5 to 10 and X is 0.3 to −1.5; and (4) testing the peptides identified in step 3 in an in vitro or in vivo assay to confirm that said peptides are cell-penetrating.

In another embodiment, the present invention relates to a cell penetrating peptide having an amino acid sequence selected from the group consisting of SEQ ID NOs: 1-455 and compositions and conjugates containing the same. In particular, the present invention relates to the cell penetrating peptides of the present invention which are conjugated to small molecules, nucleic acids, fluorescent moieties, proteins, peptides, or other cargo for delivery to the inside of cells (such as the cytoplasm or nucleus) for various therapeutic and other applications.

In other embodiments, the present invention relates to an isolated nucleotide encoding a peptide having an amino acid sequence selected from the group consisting of SEQ ID NOs: 1-455. In other embodiments, the present invention relates to a vector comprising an isolated nucleotide encoding a peptide having an amino acid sequence selected from the group consisting of SEQ ID NOs: 1-455. The present invention also relates to methods of manufacturing and using such peptides, nucleotides, and vectors.

### BRIEF DESCRIPTION OF THE DRAWINGS

FIG. 1 is a graph plotting the polarity (PP1) and hydrophobicity (PP2) of a random set of peptides extracted from natural sequences, wherein the small dots indicate random peptides, the larger dots indicate cell-penetrating peptides among the random set of peptides (according to the literature), the triangles indicate the cell-penetrating peptides of SEQ ID NOs. 1-9 among the random set of peptides (discovered to be cell-penetrating by the present inventors), and the stars indicate the cell-penetrating peptides of SEQ ID NOs. 10-19 among the random set of peptides (discovered to be cell-penetrating by the present inventors). The diagonal lines (labeled A and B) define areas to the right of each line where (according to the present invention) peptides within that area have an increased probability of being cell-penetrating. The area to the right of line A is an area that is defined when X1 is 1.7 and X is 0.3. The area to the right of line B is an area that is defined when X1 is 1.7 and X is −0.2.

FIGS. 2A-2B show the results of the cell penetration of the peptides of Examples 1-9 (SEQ ID NOs. 1-9 identified by the present invention which were covalently attached to fluorescein isothiocyanate (FITC)) in H460 cells at a concentration of 30 μm for 2 hours.

FIGS. 3A-3B show the results of the cell penetration of the peptides of Examples 10-19 (SEQ ID NOs. 10-19 identified by the present invention which were covalently attached to fluorescein isothiocyanate (FITC)) in H460 cells at a concentration of 3 μm for 2 hours.

### DETAILED DESCRIPTION OF THE INVENTION

The present invention relates to cell penetrating peptides and methods of identifying cell penetrating peptides based upon hydrophobicity and polarity.

The polarity or PP1 of a peptide is the average polarity of all the amino acids in the peptide wherein the polarity of specific amino acids are set forth in Table 1. The hydrophobicity or PP2 of a peptide is the average hydrophobicity of all the amino acids in the peptide wherein the hydrophobicity of specific amino acids are set forth in Table 1.

TABLE 1

| Amino Acids | | | Polarity | Hydrophobicity |
|---|---|---|---|---|
| Number | 1-letter code | 3-letter code | PP1 | PP2 |
| 1 | A | Ala | −0.96 | −0.76 |
| 2 | R | Arg | 0.80 | 0.63 |
| 3 | N | Asn | 0.82 | −0.57 |
| 4 | D | Asp | 1.00 | −0.89 |
| 5 | C | Cys | −0.55 | −0.47 |
| 6 | E | Glu | 0.94 | −0.54 |
| 7 | Q | Gln | 0.78 | −0.30 |
| 8 | G | Gly | −0.88 | −1.00 |
| 9 | H | His | 0.67 | −0.11 |
| 10 | I | Ile | −0.94 | −0.05 |
| 11 | L | Leu | −0.90 | 0.03 |
| 12 | K | Lys | 0.60 | 0.10 |
| 13 | M | Met | −0.82 | 0.03 |
| 14 | F | Phe | −0.85 | 0.48 |
| 15 | P | Pro | −0.81 | −0.40 |
| 16 | S | Ser | 0.41 | −0.82 |
| 17 | T | Thr | 0.40 | −0.64 |
| 18 | W | Trp | 0.06 | 1.00 |
| 19 | Y | Tyr | 0.31 | 0.42 |
| 20 | V | Val | −1.00 | −0.43 |

Most cell penetrating peptides have many hydrophobic and/or positively charged residues, but their vast sequence diversity makes it difficult to predict whether any given peptide will be cell penetrating. Cruciani et al., J. Chemometrics, 2004; 18: 146-155, proposed a set of descriptors (PP1 [polarity] and PP2 [hydrophobicity]) for each of the 20 amino acids. However, despite these descriptors no method was proposed or exists that can reasonably predict the cell penetrating properties of a peptide based upon PP1 and PP2.

Thus, in one embodiment, the present invention relates to a method of identifying cell penetrating peptides among a group of peptides by (1) determining the polarity (or “PP1”) of said peptides; (2) determining the hydrophobicity (or “PP2”) of said peptides; (3) identifying peptides within the group, wherein PP1<[(PP2*X1)+X], wherein X1 is 1.5 to 10 and X is 0.3 to −1.5; and (4) testing the peptides identified in step 3 in an in vitro or in vivo assay to confirm that said peptides are cell-penetrating.

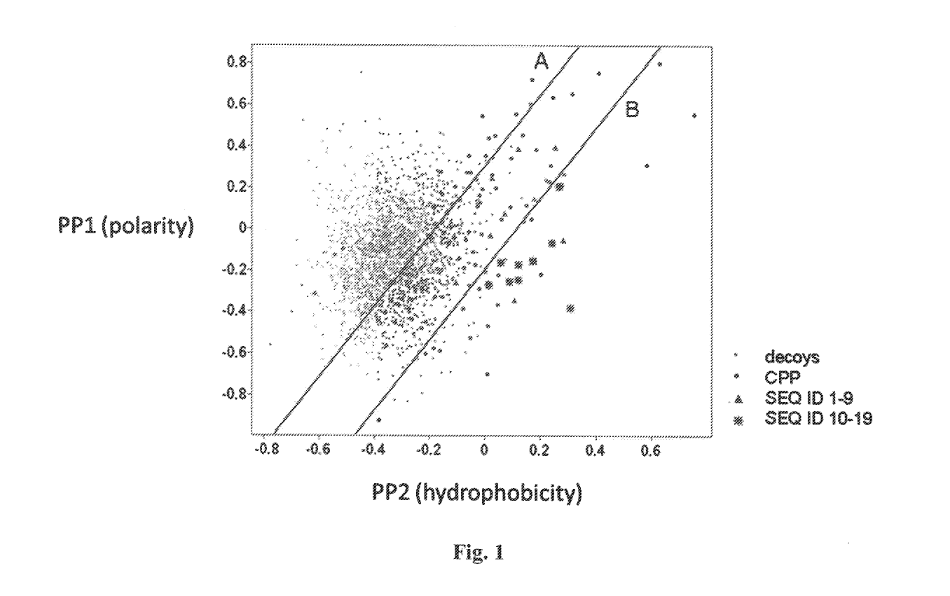

In particular embodiments, X1 is 1.7 and X is 0.3 (as shown in FIG. 1 with respect to the area to the right of line A). In other particular embodiments, X1 is 1.7 and X is −0.2 (as shown in FIG. 1 with respect to the area to the right of line B).

In other particular embodiments, X1 is 8 and X is −0.4 to 0.1. In other particular embodiments, X1 is 6 and X is −0.4 to 0.1. In other particular embodiments, X1 is 4 and X is −0.4 to 0.1. In other particular embodiments, X1 is 2 and X is −0.4 to 0.1. In other particular embodiments, X1 is 1.7 and X is −0.4 to 0.1. In other particular embodiments, X1 is 1.7 and X is 0.1. In other particular embodiments X1 is 1.7 and X is 0. In other particular embodiments, X1 is 1.7 and X is −0.1. In other particular embodiments, X1 is 1.7 and X is −0.2. In other particular embodiments, X1 is 1.7 and X is −0.3. In other particular embodiments, X1 is 1.7 and X is −0.4.

In another embodiment, the present invention relates to a cell penetrating peptide having an amino acid sequence selected from the group consisting of SEQ ID NOs: 1-455 and compositions and conjugates containing the same. In particular, the present invention relates to the cell penetrating peptides of the present invention which are conjugated to small molecules, nucleic acids, fluorescent moieties, proteins, peptides, or other cargo for delivery to the inside of cells (such as the cytoplasm or nucleus) for various therapeutic and other applications.

In other embodiments, the present invention relates to an isolated nucleotide encoding a peptide having an amino acid sequence selected from the group consisting of SEQ ID NOs: 1-455. In other embodiments, the present invention provides a vector comprising an isolated nucleotide encoding a peptide having an amino acid sequence selected from the group consisting of SEQ ID NOs: 1-455. The present invention also relates to methods of manufacturing and using such peptides, nucleotides, and vectors.

In one preferred embodiment, the present invention relates to a cell penetrating peptide having an amino acid sequence selected from the group consisting of SEQ ID NOs: 1-9 and compositions and conjugates containing the same. In another preferred embodiment, the present invention relates to a cell penetrating peptide having an amino acid sequence selected from the group consisting of SEQ ID NOs: 10, 11, 15, 16, 17 and 18 and compositions and conjugates containing the same.

In one particular embodiment, the present invention relates to a cell penetrating peptide having an amino acid sequence selected from the group consisting of SEQ ID NOs: 1-19 and compositions and conjugates containing the same.

In other particular embodiments, the present invention relates to an isolated nucleotide encoding a peptide having an amino acid sequence selected from the group consisting of SEQ ID NOs: 1-19.

In other particular embodiments, the present invention provides a vector comprising an isolated nucleotide encoding a peptide having an amino acid sequence selected from the group consisting of SEQ ID NOs: 1-19.

In other particular embodiments, the present invention relates to a cell penetrating peptide having an amino acid sequence selected from the group consisting of SEQ ID NOs: 20-30 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 20-30.

In other particular embodiments, the present invention relates to a cell penetrating peptide having an amino acid sequence selected from the group consisting of SEQ ID NOs: 31-40 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 31-40.

In other particular embodiments, the present invention relates to a cell penetrating peptide having an amino acid sequence selected from the group consisting of SEQ ID NOs: 41-50 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 41-50.

In other particular embodiments, the present invention relates to a cell penetrating peptide having an amino acid sequence selected from the group consisting of SEQ ID NOs: 51-60 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 51-60.

In other particular embodiments, the present invention relates to a cell penetrating peptide having an amino acid sequence selected from the group consisting of SEQ ID NOs: 61-70 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 61-70.

In other particular embodiments, the present invention relates to a cell penetrating peptide having an amino acid sequence selected from the group consisting of SEQ ID NOs: 71-80 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 71-80.

In other particular embodiments, the present invention relates to a cell penetrating peptide having an amino acid sequence selected from the group consisting of SEQ ID NOs: 81-90 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 81-90.

In other particular embodiments, the present invention relates to a cell penetrating peptide having an amino acid sequence selected from the group consisting of SEQ ID NOs: 91-100 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 91-100.

In other particular embodiments, the present invention relates to a cell penetrating peptide having an amino acid sequence selected from the group consisting of SEQ ID NOs: 101-110 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 101-110.

In other particular embodiments, the present invention relates to a cell penetrating peptide having an amino acid sequence selected from the group consisting of SEQ ID NOs: 111-120 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 111-120.

In other particular embodiments, the present invention relates to a cell penetrating peptide having an amino acid sequence selected from the group consisting of SEQ ID NOs: 121-130 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 121-130.

In other particular embodiments, the present invention relates to a cell penetrating peptide having an amino acid sequence selected from the group consisting of SEQ ID NOs: 131-140 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 131-140.

In other particular embodiments, the present invention relates to a cell penetrating peptide having an amino acid sequence selected from the group consisting of SEQ ID NOs: 141-150 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 141-150.

In other particular embodiments, the present invention relates to a cell penetrating peptide having an amino acid sequence selected from the group consisting of SEQ ID NOs: 151-160 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 151-160.

In other particular embodiments, the present invention relates to a cell penetrating peptide having an amino acid sequence selected from the group consisting of SEQ ID NOs: 161-170 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 161-170.

In other particular embodiments, the present invention relates to a cell penetrating peptide having an amino acid sequence selected from the group consisting of SEQ ID NOs: 171-180 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 171-180.

In other particular embodiments, the present invention relates to a cell penetrating peptide having an amino acid sequence selected from the group consisting of SEQ ID NOs: 181-190 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 181-190.

In other particular embodiments, the present invention relates to a cell penetrating peptide having an amino acid sequence selected from the group consisting of SEQ ID NOs: 191-200 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 191-200.

In other particular embodiments, the present invention relates to a cell penetrating peptide having an amino acid sequence selected from the group consisting of SEQ ID NOs: 201-210 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 201-210.

In other particular embodiments, the present invention relates to a cell penetrating peptide having an amino acid sequence selected from the group consisting of SEQ ID NOs: 211-220 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 211-220.

In other particular embodiments, the present invention relates to a cell penetrating peptide having an amino acid sequence selected from the group consisting of SEQ ID NOs: 221-230 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 221-230.

In other particular embodiments, the present invention relates to a cell penetrating peptide having an amino acid sequence selected from the group consisting of SEQ ID NOs: 231-240 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 231-240.

In other particular embodiments, the present invention relates to a cell penetrating peptide having an amino acid sequence selected from the group consisting of SEQ ID NOs: 241-250 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 241-250.

In other particular embodiments, the present invention relates to a cell penetrating peptide having an amino acid sequence selected from the group consisting of SEQ ID NOs: 251-260 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 251-260.

In other particular embodiments, the present invention relates to a cell penetrating peptide having an amino acid sequence selected from the group consisting of SEQ ID NOs: 261-270 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 261-270.

In other particular embodiments, the present invention relates to a cell penetrating peptide having an amino acid sequence selected from the group consisting of SEQ ID NOs: 271-280 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 271-280.

In other particular embodiments, the present invention relates to a cell penetrating peptide having an amino acid sequence selected from the group consisting of SEQ ID NOs: 281-290 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 281-290.

In other particular embodiments, the present invention relates to a cell penetrating peptide having an amino acid sequence selected from the group consisting of SEQ ID NOs: 291-300 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 291-300.

In other particular embodiments, the present invention relates to a cell penetrating peptide having an amino acid sequence selected from the group consisting of SEQ ID NOs: 301-310 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 301-310.

In other particular embodiments, the present invention relates to a cell penetrating peptide having an amino acid sequence selected from the group consisting of SEQ ID NOs: 311-320 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 311-320.

In other particular embodiments, the present invention relates to a cell penetrating peptide having an amino acid sequence selected from the group consisting of SEQ ID NOs: 321-330 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 321-330.

In other particular embodiments, the present invention relates to a cell penetrating peptide having an amino acid sequence selected from the group consisting of SEQ ID NOs: 331-340 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 331-340.

In other particular embodiments, the present invention relates to a cell penetrating peptide having an amino acid sequence selected from the group consisting of SEQ ID NOs: 341-350 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 341-350.

In other particular embodiments, the present invention relates to a cell penetrating peptide having an amino acid sequence selected from the group consisting of SEQ ID NOs: 351-360 and compositions and conjugates containing the same. In other embodiments, the present invention relates to a an isolated nucleotide or a vector comprising an isolated nucleotide encoding a peptide having an amino acid sequence selected from the group consisting of SEQ ID NOs 351-360.

In other particular embodiments, the present invention relates to a cell penetrating peptide having an amino acid sequence selected from the group consisting of SEQ ID NOs: 361-370 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 361-370.

In other particular embodiments, the present invention relates to a cell penetrating peptide having an amino acid sequence selected from the group consisting of SEQ ID NOs: 371-380 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 371-380.

In other particular embodiments, the present invention relates to a cell penetrating peptide having an amino acid sequence selected from the group consisting of SEQ ID NOs: 381-390 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 381-390.

In other particular embodiments, the present invention relates to a cell penetrating peptide having an amino acid sequence selected from the group consisting of SEQ ID NOs: 391-400 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 391-400.

In other particular embodiments, the present invention relates to a cell penetrating peptide having an amino acid sequence selected from the group consisting of SEQ ID NOs: 401-410 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 401-410.

In other particular embodiments, the present invention relates to a cell penetrating peptide having an amino acid sequence selected from the group consisting of SEQ ID NOs: 411-420 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 411-420.

In other particular embodiments, the present invention relates to a cell penetrating peptide having an amino acid sequence selected from the group consisting of SEQ ID NOs: 421-430 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 421-430.

In other particular embodiments, the present invention relates to a cell penetrating peptide having an amino acid sequence selected from the group consisting of SEQ ID NOs: 431-440 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 431-440.

In other particular embodiments, the present invention relates to a cell penetrating peptide having an amino acid sequence selected from the group consisting of SEQ ID NOs: 441-450 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 441-450.

In other particular embodiments, the present invention relates to a cell penetrating peptide having an amino acid sequence selected from the group consisting of SEQ ID NOs: 451-455 and compositions and conjugates containing the same. In other embodiments, the present invention relates to a an isolated nucleotide or a vector comprising an isolated nucleotide encoding a peptide having an amino acid sequence selected from the group consisting of SEQ ID NOs: 451-455.

In other particular embodiments, the present invention relates to a cell penetrating peptide wherein the PP1 of the peptide is <[(the PP2 of the peptide*X1)+X], wherein X1 is 1.7 to 2.3 and X is −0.6 to −0.85. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.6 to −0.85.

In other particular embodiments, the present invention relates to a cell penetrating peptide wherein the PP1 of the peptide is <[(the PP2 of the peptide*X1)+X], wherein X1 is 1.7 to 2.3 and X is −0.6. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.6.

In other particular embodiments, the present invention relates to a cell penetrating peptide wherein the PP1 of the peptide is <[(the PP2 of the peptide*X1)+X], wherein X1 is 1.7 to 2.3 and X is −0.65. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.65.

In other particular embodiments, the present invention relates to a cell penetrating peptide wherein the PP1 of the peptide is <[(the PP2 of the peptide*X1)+X], wherein X1 is 1.7 to 2.3 and X is −0.7. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.7.

In other particular embodiments, the present invention relates to a cell penetrating peptide wherein the PP1 of the peptide is <[(the PP2 of the peptide*X1)+X], wherein X1 is 1.7 to 2.3 and X is −0.75. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.75.

In other particular embodiments, the present invention relates to a cell penetrating peptide wherein the PP1 of the peptide is <[(the PP2 of the peptide*X1)+X], wherein X1 is 1.7 to 2.3 and X is −0.8. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.8.

In other particular embodiments, the present invention relates to a cell penetrating peptide wherein the PP1 of the peptide is <[(the PP2 of the peptide*X1)+X], wherein X1 is 1.7 to 2.3 and X is −0.85. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 1.7 to 2.3 and X is −0.85.

In other particular embodiments, the present invention relates to a cell penetrating peptide wherein the PP1 of the peptide is <[(the PP2 of the peptide*X1)+X], wherein X1 is 2.0 and X is −0.60. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.60.

In other particular embodiments, the present invention relates to a cell penetrating peptide wherein the PP1 of the peptide is <[(the PP2 of the peptide*X1)+X], wherein X1 is 2.0 and X is −0.65. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.65.

In other particular embodiments, the present invention relates to a cell penetrating peptide wherein the PP1 of the peptide is <[(the PP2 of the peptide*X1)+X], wherein X1 is 2.0 and X is −0.7. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.7.

In other particular embodiments, the present invention relates to a cell penetrating peptide wherein the PP1 of the peptide is <[(the PP2 of the peptide*X1)+X], wherein X1 is 2.0 and X is −0.75. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.75.

In other particular embodiments, the present invention relates to a cell penetrating peptide wherein the PP1 of the peptide is <[(the PP2 of the peptide*X1)+X], wherein X1 is 2.0 and X is −0.8. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.8.

In other particular embodiments, the present invention relates to a cell penetrating peptide wherein the PP1 of the peptide is <[(the PP2 of the peptide*X1)+X], wherein X1 is 2.0 and X is −0.85. In other embodiments, the present invention relates to an isolated nucleotide or a vector comprising an isolated nucleotide encoding a cell penetrating peptide wherein the PP1 of the peptide is <[(the PP2 of the peptide*X1)+X], wherein X1 is 2.0 and X is −0.85.

### General Synthesis of CPPs According to the Present Invention

All peptide sequences mentioned herein are written according to the usual convention whereby the N-terminal amino acid is on the left and the C-terminal amino acid is on the right, unless noted otherwise. A short line between two amino acid residues indicates a peptide bond. Where the amino acid has isomeric forms, it is the L form of the amino acid that is represented unless otherwise expressly indicated.

For convenience in describing this invention, the conventional and nonconventional abbreviations for the various amino acids residues are used. These abbreviations are familiar to those skilled in the art, but for clarity are listed below:

Asp-D-Aspartic Acid; Ala=A=Alanine; Arg-R=Arginine; Asn=N=Asparagine; Gly-G=Glycine; Glu=E=Glutamic Acid; Gln=Q-Glutamine; His-H=Histidine; Ile=I=Isoleucine; Leu=L=Leucine; Lys=K=Lysine; Met=M=Methionine; Phe=F=Phenylalanine; Pro=P=Proline; Ser-S-Serine; Thr=T=Threonine; Trp=W=Tryptophan; Tyr=Y=Tyrosine; and Val=V=Valine.

Also for convenience, and readily known to one skilled in the art, the following abbreviations or symbols are used to represent the moieties, reagents and the like used herein:
Et2O diethyl ether
br(s) hour(s)
TIS triisopropylsilane
Fmoc 9-fluorenylmethyloxycarbonyl
DMF dimethylformamide
DIPEA N,N-diisopropylethylamine
TFA trifluoroacetic acid
HOBT N-hydroxybenzotriazole
BOP benzotriazol-1-yloxy-tris-(dimethylamino)phosphonium-hexafluorophosphate
HBTU 2-(1H-benzotriazole-1-yl)-1,1,3,3-tetramethyluronium-hexafluorophosphate
(ES)+-LCMS electro spray liquid chromatography-mass spectrometry

In general, the peptides of the present invention may be readily synthesized by any known conventional procedure for the formation of a peptide linkage between amino acids. Such conventional procedures include, for example, any solution phase procedure permitting a condensation between the free alpha amino group of an amino acid or fragment thereof having its carboxyl group and other reactive groups protected and the free primary carboxyl group of another amino acid or fragment thereof having its amino group or other reactive groups protected.

Such conventional procedures for synthesizing the peptides of the present invention include, for example, any solid phase peptide synthesis method. In such a method the synthesis of the peptides can be carried out by sequentially incorporating the desired amino acid residues one at a time into the growing peptide chain according to the general principles of solid phase methods. Such methods are disclosed in, for example, Merrifield, R. B., J. Amer. Chem. Soc. 85, 2149-2154 (1963); Barany et al., The Peptides, Analysis, Synthesis and Biology, Vol. 2, Gross, E. and Meienhofer, J., Eds. Academic Press 1-284 (1980), which are incorporated herein by reference.

During the synthesis of peptides, it may be desired that certain reactive groups on the amino acid, for example, the alpha-amino group, a hydroxyl group, and/or reactive side chain groups, be protected to prevent a chemical reaction therewith. This may be accomplished, for example, by reacting the reactive group with a protecting group which may later be removed. For example, the alpha amino group of an amino acid or fragment thereof may be protected to prevent a chemical reaction therewith while the carboxyl group of that amino acid or fragment thereof reacts with another amino acid or fragment thereof to form a peptide bond. This may be followed by the selective removal of the alpha amino protecting group to allow a subsequent reaction to take place at that site, for example with the carboxyl group of another amino acid or fragment thereof.

Alpha amino groups may, for example, be protected by a suitable protecting group selected from aromatic urethane-type protecting groups, such as allyloxycarbony, benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); and aliphatic urethane-type protecting groups, such as t-butyloxycarbonyl (Boc), diisopropylmethyloxycarbonyl, isopropyloxycarbonyl, and allyloxycarbonyl. In an embodiment, Fmoc is used for alpha amino protection.

Hydroxyl groups (OH) of the amino acids may, for example, be protected by a suitable protecting group selected from benzyl (Bzl), 2,6-dichlorobenzyl (2,6 diCl-Bzl), and tert-butyl (t-Bu). In an embodiment wherein a hydroxyl group of tyrosine, serine, or threonine is intended to be protected, t-Bu may, for example, be used.

Epsilon-amino acid groups may, for example, be protected by a suitable protecting group selected from 2-chloro-benzyloxycarbonyl (2-Cl—Z), 2-bromo-benzyloxycarbonyl (2-Br—Z), allycarbonyl and t-butyloxycarbonyl (Boc). In an embodiment wherein an epsilon-amino group of lysine is intended to be protected, Boc may, for example, be used.

Beta- and gamma-amide groups may, for example, be protected by a suitable protecting group selected from 4-methyltrityl (Mtt), 2,4,6-trimethoxybenzyl (Tmob), 4,4′-dimethoxydityl (Dod), bis-(4-methoxyphenyl)-methyl and Trityl (Trt). In an embodiment wherein an amide group of asparagine or glutamine is intended to be protected, Trt may, for example, be used.

Indole groups may, for example, be protected by a suitable protecting group selected from formyl (For), Mesityl-2-sulfonyl (Mts) and t-butyloxycarbonyl (Boc). In an embodiment wherein the indole group of tryptophan is intended to be protected, Boc may, for example, be used.

Imidazole groups may, for example, be protected by a suitable protecting group selected from Benzyl (Bzl), t-butyloxycarbonyl (Boc), and Trityl (Trt). In an embodiment wherein the imidazole group of histidine is intended to be protected, Trt may, for example, be used.

Solid phase synthesis may be commenced from the C-terminal end of the peptide by coupling a protected alpha-amino acid to a suitable resin. Such a starting material can be prepared by attaching an alpha-amino-protected amino acid by an ester linkage to a p-benzyloxybenzyl alcohol (Wang) resin, or by an amide bond between an Fmoc-Linker, such as p-((R,S)-?-(1-(9H-fluoren-9-yl)-methoxyformamido)-2,4-dimethyloxybenzyl)-phenoxyacetic acid (Rink linker), and a benzhydrylamine (BHA) resin. Preparation of the hydroxymethyl resin is well known in the art. Fmoc-Linker-BHA resin supports are commercially available and generally used when the desired peptide being synthesized has an unsubstituted amide at the C-terminus.

In an embodiment, peptide synthesis is microwave assisted. Microwave assisted peptide synthesis is an attractive method for accelerating the solid phase peptide synthesis. This may be performed using Microwave Peptide Synthesizer, for example a Liberty peptide synthesizer (CEM Corporation, Matthews, N.C.). Microwave assisted peptide synthesis allows for methods to be created that control a reaction at a set temperature for a set amount of time. The synthesizer automatically regulates the amount of power delivered to the reaction to keep the temperature at the set point.

Typically, the amino acids or mimetic are coupled onto the Fmoc-Linker-BHA resin using the Fmoc protected form of amino acid or mimetic, with 2-5 equivalents of amino acid and a suitable coupling reagent. After coupling, the resin may be washed and dried under vacuum. Loading of the amino acid onto the resin may be determined by amino acid analysis of an aliquot of Fmoc-amino acid resin or by determination of Fmoc groups by UV analysis. Any unreacted amino groups may be capped by reacting the resin with acetic anhydride and diispropylethylamine in methylene chloride.

The resins are carried through several repetitive cycles to add amino acids sequentially. The alpha amino Fmoc protecting groups are removed under basic conditions. Piperidine, piperazine or morpholine (20-40% v/v) in DMF may be used for this purpose. In an embodiment, 20% piperidine in DMF is utilized.

Following the removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. For example, appropriate reagents for such syntheses are benzotriazol-1-yloxy-tri-(dimethylamino) phosphonium hexafluorophosphate (BOP), bromo-tris-pyrrolidino-phosphonium hexafluorophosphate (PyBroP) 2-(1H-benzotriazole-1-yl)-1,1,3,3-tetramethyluronium hexafluorophosphate (HBTU), and diisopropylcarbodiimide (DIC). In an embodiment, the reagent is HBTU or DIC. Other activating agents are described by Barany and Merrifield (in The Peptides, Vol 2, J. Meienhofer, ed., Academic Press, 1979, pp 1-284). Various reagents such as 1 hydroxybenzotriazole (HOBT), N-hydroxysuccinimide (HOSu) and 3,4-dihydro-3-hydroxy-4-oxo-1,2,3-benzotriazine (HOOBT) may be added to the coupling mixtures in order to optimize the synthetic cycles. In an embodiment, HOBT is added.

Following synthesis of the peptide, the blocking groups may be removed and the peptide cleaved from the resin. For example, the peptide-resins may be treated with 100 L ethanedithiol, 100 l dimethylsulfide, 300 L anisole, and 9.5 mL trifluoroacetic acid, per gram of resin, at room temperature for 180 min. Alternatively, the peptide-resins may be treated with 1.0 mL triisopropyl silane and 9.5 mL trifluoroacetic acid, per gram of resin, at room temperature for 90 min. The resin may then be filtered off and the peptide precipitated by addition of chilled ethyl ether. The precipitates may then be centrifuged and the ether layer decanted.

Purification of the crude peptide may be, for example, performed on a Shimadzu LC-8A system by high performance liquid chromatography (HPLC) on a reverse phase C18 Column (50×250 mm, 300 Å, 10 m). The peptides may be dissolved in a minimum amount of water and acetonitrile and injected on to a column. Gradient elution may be generally started at 2%-70% B over 70 minutes, (buffer A: 0.1% TFA/H2O, buffer B: 0.1% TFA/CH3CN) at a flow rate of 60 ml/min. UV detection set at 220/280 nm. The fractions containing the products may be separated and their purity judged on Shimadzu LC-10AT analytical system using reverse phase Pursuit C18 column (4.6×50 mm) at a flow rate of 2.5 ml/min., gradient (2-70%) over 10 min.[buffer A: 0.1% TFA/H2O, buffer B: 0.1% TFA/CH3CN)]. Fractions judged to be of high purity may then be pooled and lyophilized.

### Utility and Conjugation of the Peptides of the Present Invention

In particular embodiments, the cell penetrating peptides of the present invention (including SEQ ID NOs. 1-455) are conjugated to small molecules, nucleic acids, fluorescent moieties, proteins, peptides, or other cargo for delivery to the inside of cells (such as the cytoplasm or nucleus) for various therapeutic and other applications. Examples of such cargo include but are not limited to the cargo disclosed in U.S. Patent Application Publication No. 2008/0234183 incorporated herein by reference in its entirety. Using CPPs for delivering conjugated cargo to the inside of cells and methods of conjugating cargo such as small molecules, nucleic acids, fluorescent moieties, proteins, peptides and/or other cargo are well known in the art. See for example id. (U.S. Patent Application Publication No. 2008/0234183); Rhee et al., 201. C105Y, a Novel Cell Penetrating Peptide Enhances Gene Transfer of Sec-R Targeted Molecular Conjugates, Molecular Therapy (2005) 11, S79-S79; Johnson et al., Cell-penetrating Peptide for Enhanced Delivery of Nucleic Acids and Drugs to Ocular Tissues Including Retina and Cornea, Molecular Therapy (2007) 16 (1), 107-114; El-Andaloussi et al., A Novel Cell-penetrating Peptide, M918, for Efficient Delivery of Proteins and Peptide Nucleic Acids, Molecular Therapy (2007) 15 (10), 1820-1826; and Crombez et al., A New Potent Secondary Amphipathic Cell-Penetrating Peptide for siRNA Delivery Into Mammalian Cells, Molecular Therapy (2008) 17 (1), 95-103; Sasaki, Y. et al., Cell-penetrating peptide-conjugated XIAP-inhibitory cyclic hexapeptides enter into Jurkat cells and inhibit cell proliferation FEBS Journal (2008) 275 (23), 6011-6021; Kolluri, S. K. et al., A Short Nur77-Derived Peptide Converts Bcl-2 from a Protector to a Killer, Cancer Cell (2008) 14 (4), 285-298; Avbelj, M., The Role of Intermediary Domain of MyD88 in Cell Activation and Therapeutic Inhibition of TLRs J. Immunology (2011), 1; 187(5):2394-404.

In addition, the foregoing examples demonstrate the conjugation of SEQ ID NOs. 1-19 to fluorescein isothiocyanate (FITC) and their subsequent cell penetration as summarized in the cell assay section (also below).

### EXAMPLES

The peptides in the specific examples below were prepared by solid state synthesis. See Steward and Young, Solid Phase Peptide Synthesis, Freemantle, San Francisco, Calif. (1968). A preferred method is the Merrifield process. Merrifield, Recent Progress in Hormone Res., 23:451 (1967). In addition, the peptides in the specific examples below were synthesized by tagging the N-terminus of the peptide with FITC as a green fluorescent dye. Examples 1-9 were prepared by C S Bio Company, Inc. and Examples 10-19 were prepared by HYBIO Pharmaceutical Co., Ltd.

### Example 1: Synthesis of FITC-6Ahx-MWQPRRPWPRVPWRW-NH2

Material:

All chemicals and solvents such as DMF (Dimethylformamide), DCM (Methylene Chloride), DIEA (Diisopropylethylamine), and piperidine were purchased from VWR and Aldrich, and used as purchased without further purification. Mass spectra were recorded with Electrospray ionization mode. The automated stepwise assembly of protected amino acids was constructed on a CS 336X series peptide synthesizer (C S Bio Company, Menlo Park, Calif., USA) with Rink Amide MBHA resin as the polymer support. N-(9-fluorenyl)methoxycarbonyl (Fmoc) chemistry was employed for the synthesis. The protecting groups for Fmoc amino acids (AAs) were as follows, Arg: (Pbf), Asn/Gln/Cys/His: (Trt), Asp/Glu: (OtBu), Lys/Trp: (Boc), Ser/Thr/Tyr: (tBu).

Synthesis:

The above peptide (SEQ ID NO. 1) as conjugated to FITC was synthesized using Fmoc chemistry. The synthesis route started from deFmoc of pre-loaded Rink Amide resin and coupling/de-protecting of desired AAs according to the given sequences for all the orders. Coupling reagent was DIC/HOBt, and reaction solvents were DMF and DCM. The ratio of peptidyl resin/AA/DIC/HOBT was 1/4/4/4 (mol/mol). After coupling program, DeFmoc was executed using 20% piperidine in DMF. For example, a 0.4 mmol synthesis was performed till the last AA was attached. After deFmoc, the resin was coupled with Fmoc-Ahx-OH, followed by deFmoc and FITC attachment.

Fmoc-Rink Amide Resin (0.85 g, 0.4 mmol, sub: 0.47 mm/g, Lot#110810, C S Bio) was mixed in a 25 mL reaction vessel (RV) with DMF (10 mL), and swollen for 10-30 min. The RV was mounted on a CS336 peptide automated synthesizer and the amino acids were loaded onto amino acid (AA) wheel according to the given peptide sequence. HOBt (0.5M in DMF) and DIC (0.5M in DMF) were all pre-dissolved separately in transferable bottles under N2. Fmoc-amino acids (AAs, 4 eq) were weighed and prelocated as powder on the AA wheel. For example, 0.4 mmol synthesis needed 1.6 mmol of AA. The preset program started from AA dissolving in the AA tube and the solution was pumped thru M-VA to T-VA. HOBt solution was later mixed with AA. N2 bubbling was used to assist mixing. While DIC solution was combined with the AA/HOBt solution, the whole mixture was transferred into the RV with drained resin in 5 min and the coupling started at the same time.

After shaking for 3-6 hr, reaction mixture was filtered off and the resin was washed with DMF three times, followed by deFmoc according to the preset program using 20% Pip in DMF. The next AA was attached following the same route. Seven washing steps were done with DMF/DCM alternatively after deFmoc. The coupling process was repeated with the respective building blocks according to the given sequence till the last AA was coupled. Coupling Time: 3-6 hrs for each AA attachment. After deFmoc of last AA, the resin was coupled with Fmoc-Ahx-OH (3 eq) using DIC/HOBt. After deFmoc, FITC (3 eq) was attached in DMF with 1-2 eq of DIEA.

Cleavage:

The final peptidyl resin (1-1.5 g) was mixed with TFA cocktail (TFA/EDT/TIS/H2O) and the mixture was shaken at room temperature for 4 hr. The cleaved peptide was filtered and the resin was washed by TFA. After ether precipitation and washing, the crude peptide was obtained in a yield of 50-90%. The crude peptide was directly purified without lyophilization.

Purification:

100 mg of FITC peptide were dissolved in Buffer A 0.1% TFA in water and ACN, and the peptide solution was loaded onto a C18 column (2 inch) with a prep HPLC purification system. With a flow rate of 25-40 mL/min, the purification was finished in a TFA (0.1%) buffer system with a 60 min gradient. Fractions (peptide purity>95%) containing the expected MW were collected. The prep HPLC column was then washed for at least three void column volumes by 80% Buffer B and equilibrated to 5% Buffer B before next loading.

Lyophilzation:

The fractions (purity>90%) were combined and transferred to 1 L lyophilization jars which were deeply frozen by liquid nitrogen. After freezing, the jars were placed onto Lyophilizer (Virtis Freezemobile 35EL) and dried overnight. The vacuum was below 500 mT and chamber temperature was below −60° C. The lyophilisation was completed in 12-18 hrs at room temperature (environment temperature).

Results:

Starting from 0.2 mm synthesis, purification was done in a TFA system and the final yield was 15 mg (2.8%) of product. (ES)+-LCMS m/e calculated (“calcd”) for C130H167N35O22S2 found 2636.1.

### Example 2: Synthesis of FITC-6Ahx-LRLLHRRQKRIIGGK-NH2

The above peptide (SEQ ID NO. 2) as conjugated to FITC was synthesized using Fmoc chemistry. Fmoc Rink Amide MBHA resin (0.2 mmol) was subjected to solid phase synthesis and purification by following the procedure in example 1 to yield 19 mg (4.0%) of the above peptide. (ES)+-LCMS m/c calculated (“calcd”) for C108H173N35O22S found 2345.84.

### Example 3: Synthesis of FITC-6Ahx-RQHGLRHFYNRRRRS-NH2

The above peptide (SEQ ID NO. 3) as conjugated to FITC was synthesized using Fmoc chemistry. Fmoc Rink Amide MBHA resin (0.2 mmol) was subjected to solid phase synthesis and purification by following the procedure in example 1 to yield 17 mg (3.3%) of the above peptide. (ES)+-LCMS m/e calculated (“calcd”) for C113H162N42O25S found 2540.86.

### Example 4: Synthesis of FITC-6Ahbx-KLWKKKELLQRAEKKKKIKK-NH2

The above peptide (SEQ ID NO. 4) as conjugated to FITC was synthesized using Fmoc chemistry. Fmoc Rink Amide MBHA resin (0.2 mmol) was subjected to solid phase synthesis and purification by following the procedure in example 1 to yield 52 mg (8.5%) of the above peptide. (ES)+-LCMS m/e calculated (“calcd”) for C146H238N38O31S found 3053.79.

### Example 5: Synthesis of FITC-6Ahx-MPKFKQRRRKLKAKAERLFK-NH2

The above peptide (SEQ ID NO. 5) as conjugated to FITC was synthesized using Fmoc chemistry. Fmoc Rink Amide MBHA resin (0.2 mmol) was subjected to solid phase synthesis and purification by following the procedure in example 1 to yield 75 mg (12.2%) of the above peptide. (ES)+-LCMS m/e calculated (“calcd”) for C143H226N42O29S2 found 3061.76.

### Example 6: Synthesis of FITC-6Ahx-FVFPRLRD R-TLAMAARKASRNH2

The above peptide (SEQ ID NO. 6) as conjugated to FITC was synthesized using Fmoc chemistry. Fmoc Rink Amide MBHA resin (0.2 mmol) was subjected to solid phase synthesis and purification by following the procedure in example 1 to yield 12 mg (2.1%) of the above peptide. (ES)+-LCMS m/e calculated (“calcd”) for C134H196N36O30S2 found 2855.38.

### Example 7: Synthesis of FITC-6Ahx-YLKFIPLKRAIWLIK-NH2

The above peptide (SEQ ID NO. 7) as conjugated to FITC was synthesized using Fmoc chemistry. Fmoc Rink Amide MBHA resin (0.2 mmol) was subjected to solid phase synthesis and purification by following the procedure in example 1 to yield 15 mg (3.1%) of the above peptide. (ES)+-LCMS m/e calculated (“calcd”) for C124H179N25S22S found 2404.

### Example 8: Synthesis of FITC-6Abx-KRKRPFVLKKKRGRKRRRI-NH2

The above peptide (SEQ ID NO. 8) as conjugated to FITC was synthesized using Fmoc chemistry. Fmoc Rink Amide MBHA resin (0.2 mmol) was subjected to solid phase synthesis and purification by following the procedure in example 1 to yield 78 mg (12.5%) of the above peptide. (ES)+-LCMS m/e calculated (“calcd”) for C144H242N50O26S found 3121.89.

### Example 9: Synthesis of FITC-6Ahx-RTTRRWKRWFKFRKRKGEKR-NH2

The above peptide (SEQ ID NO. 9) as conjugated to FITC was synthesized using Fmoc chemistry. Fmoc Rink Amide MBHA resin (0.2 mmol) was subjected to solid phase synthesis and purification by following the procedure in example 1 to yield 17 mg (2.6%) of the above peptide. (ES)+-LCMS m/e calculated (“calcd”) for C154H231N51O30S found 3308.91.

### Example 10: Synthesis of FITC-6Ahx-MVLKFFRWLFRLLFR-NH2

The above peptide (SEQ ID NO. 10) as conjugated to FITC was synthesized using Fmoc chemistry. The synthesis was carried out on a 0.15 mmole scale using the Fmoc-Linker-Rink amide resin (0.5 g, Sub=0.3 mmol/g). 0.5 g dry resin was placed in a peptide synthesis reactor column (20×150 mm), swelled and washed with DMF. 20% piperidine was then added, agitated for 5 min and drained, then, 20% piperidine was added again, agitated for 7 min, and then the resin was washed with DMF. 0.75 mmol (5 eq) Fmoc-Arg(Pbf)-OH, 0.75 mmol HOBt, 0.75 mmol HBTU, and 0.75 mmol DIPEA were added into the reaction column, and agitated gently for 2 hours with nitrogen. Some resin sample was subjected to a color test, and then the Fmoc group was deprotected. The steps above were repeated until all the amino acids were coupled. At the end of the synthesis, the resin was transferred to a reaction vessel on a shaker for cleavage. The peptide was cleaved from the resin using a 20.0 mL cleavage cocktail (TFA:TIS:H2O:EDT=91:3:3:3(v/v)) for 120 minutes at room temperature avoiding light. The deprotection solution was added to 1000 mL cold Et2O to precipitate the peptide. The peptide was centrifuged in 250 mL polypropylene tubes. The precipitates from the individual tubes were combined in a single tube and washed 3 times with cold Et2O and dried in a desiccator under house vacuum.

The crude material was purified by preparative HPLC on a C18-Column (250×46 mm, 10?m particle size) and eluted with a linear gradient of 5-95% B (buffer A: 0.1% TFA/H2O; buffer B:ACN) in 30 min., with a flow rate 19 mL/min, with detection at 220 nm. The fractions were collected and were checked by analytical HPLC. Fractions containing pure product were combined and lyophilized to a white amorphous powder.

FITC coupling: 0.15 mmol of peptidyl resin was placed in the reaction vessel, followed by addition of 0.165 mmol FITC, with a reagent mixture of Pyridine:DMF:DCM=12:7:5 (V/V). The mixture was reacted for 2 hours in N2. After that, the peptide was cleaved from the resin.

The yield was 80 mg (18%) of the above peptide. (ES)+-LCMS m/e calculated (“calcd”) for C132H181N29O21 S2 found 2574.78.

### Example 11: Synthesis of FITC-6Ahx-RLWEFYKLYKRRHRV-NH2

The above peptide (SEQ ID NO. 11) as conjugated to FITC was synthesized using Fmoc chemistry. Fmoc Rink Amide MBHA resin (0.15 mmol) was subjected to solid phase synthesis and purification by following the procedure in example 10 to yield 90 mg (18%) of the above peptide. (ES)+-LCMS m/e calculated (“calcd”) for C129H179N35O25S found 2652.12.

### Example 12: Synthesis of FITC-6Ahx-KVFSPKKKMEFFLLF-NH2

The above peptide (SEQ ID NO. 12) as conjugated to FITC was synthesized using Fmoc chemistry. Fmoc Rink Amide MBHA resin (0.15 mmol) was subjected to solid phase synthesis and purification by following the procedure in example 10 to yield 50 mg (12%) of the above peptide. (ES)+-LCMS m/e calculated (“calcd”) for C122H168N22O24S2 found 2389.5.

### Example 13: Synthesis of FITC-6Ahz-VKIWFQNRRVRWRKR-NH2

The above peptide (SEQ ID NO. 13) as conjugated to FITC was synthesized using Fmoc chemistry. Fmoc Rink Amide MBHA resin (0.15 mmol) was subjected to solid phase synthesis and purification by following the procedure in example 10 to yield 60 mg (12%) of the above peptide. (ES)+-LCMS m/e calculated (“calcd”) for C125H181N39O23 S found 2630.12.

### Example 14: Synthesis of FITC-6Ahx-MRMIRFRKKIPYLRY-NH2

The above peptide (SEQ ID NO. 14) as conjugated to FITC was synthesized using Fmoc chemistry. Fmoc Rink Amide MBHA resin (0.15 mmol) was subjected to solid phase synthesis and purification by following the procedure in example 10 to yield 55 mg (11%) of the above peptide. (ES)+-LCMS m/e calculated (“calcd”) for C123H182N32O23S3 found 2573.6.

### Example 15: Synthesis of FITC-6Ahx-PKWTRPLLPFWKRYL-NH2

The above peptide (SEQ ID NO. 15) as conjugated to FITC was synthesized using Fmoc chemistry. Fmoc Rink Amide MBHA resin (0.15 mmol) was subjected to solid phase synthesis and purification by following the procedure in example 10 to yield 50 mg (11%) of the above peptide. (ES)+-LCMS m/e calculated (“calcd”) for C128H172N28O23S found 2501.7.

### Example 16: Synthesis of FITC-6Ahx-RWFAFKMMMAKKWAK-NH2

The above peptide (SEQ ID NO. 16) as conjugated to FITC was synthesized using Fmoc chemistry. Fmoc Rink Amide MBHA resin (0.15 mmol) was subjected to solid phase synthesis and purification by following the procedure in example 10 to yield 20 mg (4%) of the above peptide. (ES)+-LCMS m/e calculated (“calcd”) for C121H165N27O21S found 2461.6.

### Example 17: Synthesis of FITC-6Ahx-SKIVRVIFRYAKWLF-NH2

The above peptide (SEQ ID NO. 17) as conjugated to FITC was synthesized using Fmoc chemistry. Fmoc Rink Amide MBHA resin (0.15 mmol) was subjected to solid phase synthesis and purification by following the procedure in example 10 to yield 25 mg (6%) of the above peptide. (ES)+-LCMS m/e calculated (“calcd”) for C123H171N27O23S found 2427.8.

### Example 18: Synthesis of FITC-6Ahx-KFFKLKHFILNILKQ-NH2

The above peptide (SEQ ID NO. 18) as conjugated to FITC was synthesized using Fmoc chemistry. Fmoc Rink Amide MBHA resin (0.15 mmol) was subjected to solid phase synthesis and purification by following the procedure in example 10 to yield 80 mg (19%) of the above peptide. (ES)+-LCMS m/e calculated (“calcd”) for C123H176N26O23S found 2417.8.

### Example 19: Synthesis of FITC-6Ahx-LLPQWPRIRHIKLLR-NH2

The above peptide (SEQ ID NO. 19) as conjugated to FITC was synthesized using Fmoc chemistry. Fmoc Rink Amide MBHA resin (0.15 mmol) was subjected to solid phase synthesis and purification by following the procedure in example 10 to yield 90 mg (21%) of the above peptide. (ES)+-LCMS m/e calculated (“calcd”) for C119H178N32O22 S found 2439.8.

### Example 20: Cell Assays

The peptides of Examples 1-19 were tested for cell penetration in H460 and HeLa cell lines as follows.

Materials:

The H460 cell line and HeLa (ATCC) were maintained in growth media then passaged every 2-3 days. Growth media for H460 was RPMI 1640, 10% fetal calf serum, sodium pyruvate, antibiotics and glutamine (GIBCO). Growth media for HeLa cells was DMEM supplemented with 10% heat-inactivated fetal calf serum, antibiotics and glutamine (GIBCO).

Methods and Procedures:

Cells were plated onto Whatman glass-bottom 96-well plates or Perkin Elmer glass-bottom 96-well plates and cultured overnight. Peptide stocks were prepared in DMSO and were diluted in cell growth media for cellular uptake studies. After 2 and 24 h of peptide incubation at various concentrations, media was removed followed by three washes of acidic saline. Formaldehyde fixation, with or without Hoechst 33342 dye solution (to stain nuclei), was followed by PBS washes. Plates were imaged on the Operetta High Content Imaging system in confocal fluorescence mode using the 40× water immersion high NA objective.

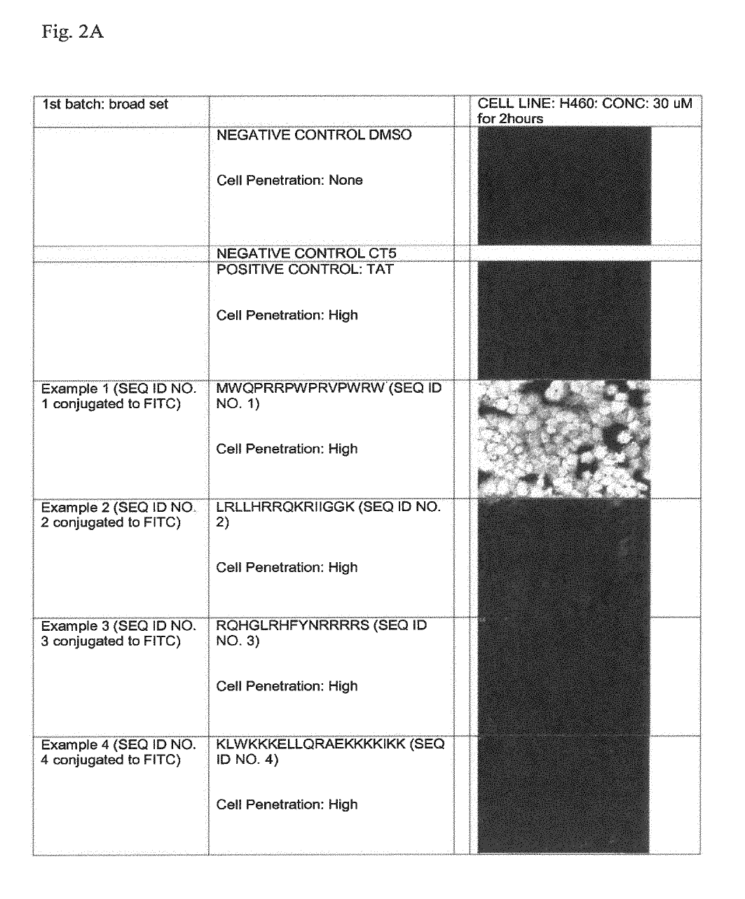

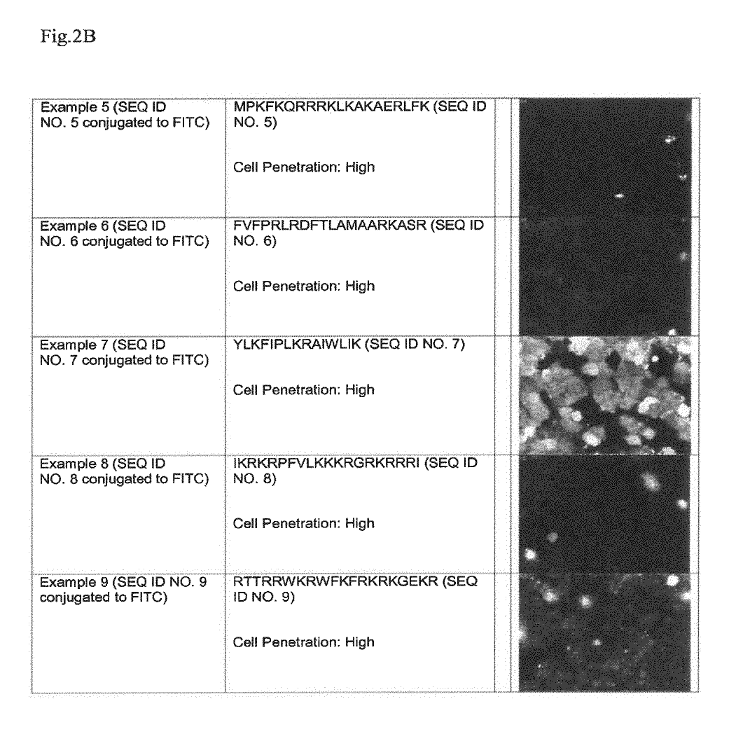

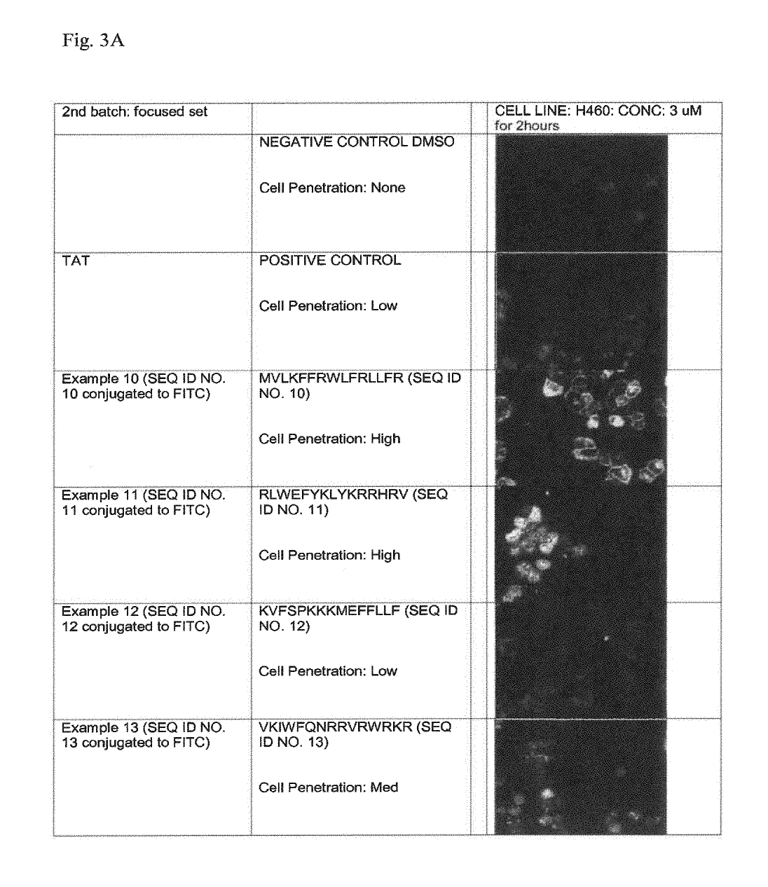

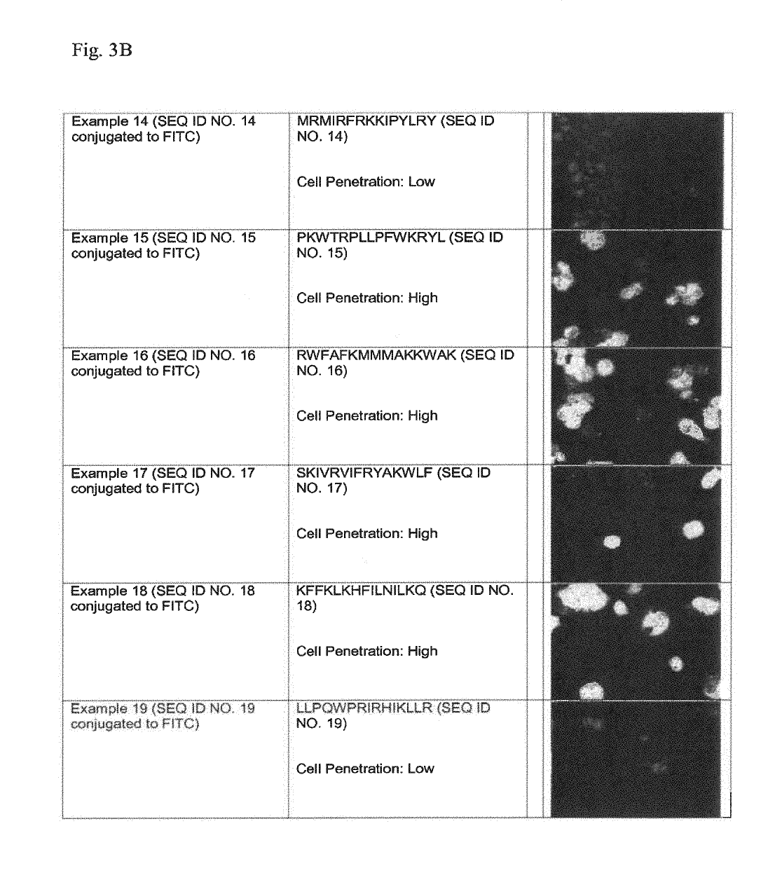

The results for the peptides of Examples 1-9 in H460 cells are shown in FIGS. 2A and 2B. As shown in the Figures, the cell penetration as determined by the fluorescence for the peptides of Examples 1-9 (SEQ ID NOS. 1-9) was high. The results for the peptides of Examples 10-19 in H460 cells are shown in FIGS. 3A and 3B which varied but which all showed some cell penetration. For example, the cell penetration for the peptides of Examples 10-11 and 15-18 (SEQ ID NOS. 10-11 and 15-18, respectively) were high. The cell penetration for the peptide of Example 13 (SEQ ID NO. 13) was medium and the cell penetration for the peptides of Examples 12, 14, and 19 (SEQ ID NOS. 12, 14, and 19) were low but still cell penetrating. The results in the HeLA cells were similar.

### Example 21 Identification of Additional Peptides Predicted to be Cell Penetrating

Using the method of the present invention, additional peptides were identified that are predicted to be cell-penetrating. For example, the peptides of SEQ ID NOS. 20-455 are peptides wherein PP1<[(PP2*X1)+X], wherein X1 is 1.5 to 10 and X is 0.3 to −1.5, and therefore are predicted to be cell-penetrating. See Table 2.

Table 2 shows the peptides of SEQ ID NOS. 20-455 identified within larger sequences or proteins which are predicted to be cell-penetrating according to the method of the present invention of identifying cell penetrating peptides.

TABLE 2
Further cell penetrating peptides of the invention

| SEQ | | | | |
|---|---|---|---|---|
| ID | | | HYDRO- | |
| No. | Sequence | | PHOBICITY | POLARITY |
| 20 | AARLWFF | Urease accessory protein ureD | 0.33500 | -0.17500 |
| | RLWRR | | | |
| 21 | AFFILKW | Prolipoprotein diacylglyceryl | 0.21750 | -0.24000 |
| | KLWKK | transferase | | |
| 22 | AFLFRRF | Probable RNA-directed RNA | 0.27000 | -0.06250 |
| | YDRRF | polymerase | | |
| 23 | AFRFIKRL | Leucyl-tRNA synthetase | 0.23083 | -0.27833 |
| | WRLV | | | |
| 24 | AIVLYFFC | Prolipoprotein diacylglyceryl | 0.13500 | -0.35333 |
| | RRRL | transferase | | |
| 25 | ALFFAWK | Mercuric transport protein | 0.15000 | -0.30833 |
| | RIYRP | | | |
| 26 | ALFFAWR | Mercuric transport protein | 0.12333 | -0.40083 |
| | RIVRP | | | |
| 27 | ALFFAWR | Mercuric transport protein | 0.19417 | -0.29167 |
| | RIYRP | | | |
| 28 | ALICFLIF | Protein AXL2 | 0.21500 | -0.36917 |
| | WRRR | | | |
| 29 | AWAVMA | Cobalamin synthase | 0.22417 | -0.24750 |
| | RWFWRR | | | |
| 30 | AWRFLGR | Leucyl-tRNA synthetase | 0.15083 | -0.33083 |
| | VWRLV | | | |
| 31 | AWRLRK | Putative uncharacterized protein | 0.27833 | -0.05917 |
| | NFFYFY | LOC644538 | | |
| 32 | CRFIMRC | Protein 3 | 0.20333 | -0.23667 |
| | WLCWK | | | |
| 33 | CRLLWIF | Leucine-rich repeat and | 0.43083 | -0.00167 |
| | RRRWR | immunoglobulin-like domain- | | |
| | | containing nogo receptor- | | |
| | | interacting protein 1 | | |
| 34 | FAFRFAF | Cobalamin synthase | 0.17917 | -0.29667 |
| | KRWLT | | | |
| 35 | FALILIFR | Prolipoprotein diacylglyceryl | 0.21833 | -0.29000 |
| | RKWK | transferase | | |
| 36 | FCGFLWF | Magnesium transporter MRS2-B | 0.22750 | -0.28000 |
| | FKYKR | | | |
| 37 | FFALRYI | Envelope glycoprotein B | 0.14917 | -0.37250 |
| | MRLRA | | | |
| 38 | FFCFFRKR | Uncharacterized membrane protein | 0.29250 | -0.24917 |
| | WKVL | C2G11.09 | | |
| 39 | FFCWAW | Golgin subfamily A member 8-like | 0.32333 | -0.13333 |
| | LPRRRR | protein 1 | | |
| 40 | FFFFKCRR | Putative uncharacterized protein | 0.32083 | -0.33250 |
| | WLCF | YKL030W | | |
| 41 | FFFLRRFE | Splicing factor, arginine/serine- | 0.31500 | -0.21917 |
| | RGFW | rich 8 | | |
| 42 | FFFVARD | Probable potassium transport | 0.29117 | -0.17417 |
| | LWKWR | system protein kup | | |
| 43 | FFFWKIRP | ATP synthase subunit b | 0.18667 | -0.26333 |
| | QIAR | | | |
| 44 | FFFWKIYP | ATP synthase subunit b | 0.24083 | -0.17417 |
| | QIRK | | | |
| 45 | FFILKRLN | Ammonium transporter 1 member 3 | 0.14750 | -0.34667 |
| | LLRI | | | |
| 46 | FFIRLFRK | Phospho-N-acetylmuramoyl- | 0.14417 | -0.40250 |
| | IGWG | pentapeptide-transferase | | |
| 47 | FFIRRLRL | Transport protein particle 130 kDa | 0.20667 | -0.19167 |
| | LKLE | subunit | | |
| 48 | FFKRLPK | Late 100 kDa protein | 0.16917 | -0.27250 |
| | WRLGI | | | |
| 49 | FFLKRKM | Putative membrane protein ycf1 C- | 0.20000 | -0.20667 |
| | KEFLF | terminal part | | |
| 50 | FFLQMAV | Abnormal spindle-like | 0.19333 | -0.22833 |
| | YRRRF | microcephaly-associated protein | | |
| | | homolog | | |
| 51 | FFMYYFL | Uncharacterized protein ORF149 | 0.30000 | -0.06417 |
| | WKKNR | | | |
| 52 | FFRFLLRK | Vitamin K-dependent gamma- | 0.28000 | -0.38250 |
| | LYVF | carboxylase | | |
| 53 | FFRLFRVL | Voltage-dependent L-type calcium | 0.22167 | -0.35417 |
| | RLVK | channel subunit alpha-1S | | |
| 54 | FFRLFRV | Voltage-dependent L-type calcium | 0.25333 | -0.34250 |
| | MRLIK | channel subunit alpha-1S | | |
| 55 | FFRLFRV | Voltage-dependent L-type calcium | 0.22167 | -0.34750 |
| | MRLVK | channel subunit alpha-1C | | |
| 56 | FFRLFRV | Voltage-dependent L-type calcium | 0.22167 | -0.34750 |
| | MRLVK | channel subunit alpha-1D | | |
| 57 | FFRYILKR | Regulatory protein BlaR1 | 0.28917 | -0.03667 |
| | YFNY | | | |
| 58 | FGAFLKR | Probable kinetochore protein spc25 | 0.14667 | -0.33417 |
| | MRRLF | | | |
| 59 | FGRFYRG | Maturase K | 0.22250 | -0.18500 |
| | RIWYL | | | |
| 60 | FIGILFRIL | Hereditary hemochromatosis | 0.15500 | -0.35000 |
| | RKR | protein homolog | | |
| 61 | FILMKKW | Maturase k | 0.14667 | -0.31083 |
| | KFHLV | | | |
| 62 | FILWIKRI | Activated factor Xa heavy chain | 0.24000 | -0.28583 |
| | MRLK | | | |
| 63 | FIRRIFRR | Capsid protein | 0.23750 | -0.21167 |
| | LPTF | | | |
| 64 | FIRRIFRR | Capsid protein | 0.23750 | -0.21167 |
| | LPTF | | | |
| 65 | FITWLKL | Uncharacterized protein ycf54 | 0.21750 | -0.20500 |
| | RLRYI | | | |
| 66 | FKAFFIRR | Uncharacterized | 0.24500 | -0.38667 |
| | YFVF | glycosyltransferase RF_0337 | | |
| 67 | FKFFFRR | Testis-specific Y-encoded-like | 0.32000 | -0.07750 |
| | NPYFR | protein 1 | | |
| 68 | FKKLIPW | Uncharacterized membrane protein | 0.17167 | -0.29583 |
| | FSFRM | epsK | | |
| 69 | FKRILLNI | Probable cytochrome P450 515A1 | 0.18000 | -0.24583 |
| | LYRF | | | |
| 70 | FKRIPWFI | UDP-2,3-diacylglucosamine | 0.24750 | -0.15583 |
| | KKRI | hydrolase | | |
| 71 | FKVGLW | Glycosylphosphatidylinositol | 0.14917 | -0.32917 |
| | KRYFIL | anchor biosynthesis protein 11 | | |
| 72 | FLALPLRL | UDP-2,3-diacylglucosamine | 0.15917 | -0.33000 |
| | RRRI | hydrolase | | |
| 73 | FLAMPLR | UDP-2,3-diacylglucosamine | 0.14583 | -0.40167 |
| | WRLKI | hydrolase | | |
| 74 | FLFFKGK | Processed glycerol phosphate | 0.15917 | -0.33083 |
| | KAYWF | lipoteichoic acid synthase | | |
| 75 | FLFLKWR | Transient receptor potential channel | 0.37833 | -0.13583 |
| | RIRKF | pyrexia | | |
| 76 | FLFPRRR | Ethylene-responsive transcription | 0.23000 | -0.20417 |
| | VKRLI | factor CRF4 | | |
| 77 | FLFRVFR | Fanconi anemia group A protein | 0.21083 | -0.19417 |
| | RRLQA | homolog | | |
| 78 | FLILRIKL | Uncharacterized protein RSN1 | 0.19167 | -0.27167 |
| | KRIY | | | |
| 79 | FLIVRMR | Nucleoside diphosphate kinase 6 | 0.20583 | -0.24250 |
| | ELLWR | | | |
| 80 | FLIYKFKR | VPS10 domain-containing receptor | 0.23667 | -0.19333 |
| | KIPW | SorCS3 | | |
| 81 | FLKFPFLK | Uncharacterized metalloprotease | 0.20000 | -0.26583 |
| | KYRI | bbp_296 | | |
| 82 | FLKLYVLI | Mediator of RNA polymerase II | 0.15583 | -0.30583 |
| | KWCR | transcription subunit 14 | | |
| 83 | FLKRYLL | Putative membrane protein ycf1 | 0.29667 | -0.16250 |
| | FQLRW | | | |
| 84 | FLKRYLL | Putative membrane protein ycfl | 0.29667 | -0.16250 |
| | FQLRW | | | |
| 85 | FLLAAYF | Receptor-type tyrosine-protein | 0.18667 | -0.38417 |
| | FRFRK | phosphatase epsilon | | |
| 86 | FLLCYWK | Tumor necrosis factor receptor | 0.21833 | -0.17333 |
| | ACWRR | superfamily member 8 | | |
| 87 | FLLIRRVL | Protein SIP3 | 0.23750 | -0.28083 |
| | RYYL | | | |
| 88 | FLLLKVF | Probable integrase/recombinase | 0.16667 | -0.39917 |
| | YRVLR | protein MJ0367 | | |
| 89 | FLLLLLFL | EP-cadherin | 0.17250 | -0.37500 |
| | KRKK | | | |
| 90 | FLLPWRR | B1 bradykinin receptor | 0.36917 | 0.05667 |
| | WWQQR | | | |
| 91 | FLLRRGIY | Alanyl-tRNA synthetase | 0.17250 | -0.29000 |
| | RAWM | | | |
| 92 | FLLSMRY | NADH-quinone oxidoreductase | 0.17417 | -0.25500 |
| | FFRPK | subunit 11 | | |
| 93 | FLMKKW | Maturase K | 0.21750 | -0.20167 |
| | KYFLIH | | | |
| 94 | FLMLLRR | Amiloride-sensitive sodium | 0.26667 | -0.10833 |
| | FRSRY | channel subunit aloha | | |
| 95 | FLRFVLR | Vitamin K-dependent gamma- | 0.20417 | -0.39500 |
| | KLYVF | carboxylase | | |
| 96 | FLRFVLR | Vitamin K-dependent gamma- | 0.20417 | -0.39500 |
| | KLYVF | carboxylase | | |
| 97 | FLRLFRA | Probable voltage-dependent N-type | 0.12250 | -0.35500 |
| | ARLIK | calcium channel subunit alpha-1B | | |
| 98 | FLRYLSW | 50S ribosomal protein L32e | 0.37167 | -0.10917 |
| | RFWKF | | | |
| 99 | FLTLPLFI | UDP-2,3-diacylglucosamine | 0.15000 | -0.35750 |
| | RRRI | hydrolase | | |
| 100 | FLWIPLRL | UDP-2,3-diacylglucosamine | 0.24917 | -0.39000 |
| | RLRI | hydrolase | | |
| 101 | FLWLPLR | UDP-2,3-diacylglucosamine | 0.29333 | -0.38250 |
| | FRLRI | hydrolase | | |
| 102 | FLYFRRTP | DNA translocase ftsK | 0.19750 | -0.23833 |
| | RPLF | | | |
| 103 | FMFLFFL | Prolipoprotein diacylglyceryl | 0.33083 | -0.38083 |
| | WRKPR | transferase | | |
| 104 | FMWVRW | NADH-quinone oxidoreductase | 0.28667 | -0.19250 |
| | TLPRFR | subunit H 1 | | |
| 105 | FPWRKFP | Uncharacterized 16.5 kDa protein | 0.22000 | -0.17083 |
| | RYLKV | in 100 kDa protein region | | |
| 106 | FPWSFRL | Transposase for transposon gamma- | 0.21750 | -0.18583 |
| | FRLLY | delta | | |
| 107 | FQLFFRRF | Protein translocase subunit secA 3 | 0.23167 | -0.20917 |
| | LRLS | | | |
| 108 | FRFRFWR | Calpain-5 | 0.37333 | -0.18000 |
| | FGKWV | | | |
| 109 | FRGLFRFL | ATP-dependent helicase/nuclease | 0.19000 | -0.30667 |
| | RFIE | subunit A | | |
| 110 | FRKFPWY | Uncharacterized membrane protein | 0.19583 | -0.21500 |
| | KVPIY | C977.17 | | |
| 111 | FRKRMM | Splicing factor 4 | 0.27750 | -0.09083 |
| | LAYRFR | | | |
| 112 | FRMKLRN | RRP 12-like protein | 0.19750 | -0.20750 |
| | LFIKF | | | |
| 113 | FRPLAPRP | Proprotein convertase | 0.20583 | -0.29333 |
| | WRWL | subtilisin/kexin type 6 | | |
| 114 | FRRFFTR | Na(+)/H(+) antiporter subunit E | 0.36000 | -0.02917 |
| | QFYLW | | | |
| 115 | FRRFFYR | Protein COS8 | 0.26000 | -0.12750 |
| | LLSLK | | | |
| 116 | FRRFVWN | Xenotropic and polytropic | 0.27500 | -0.09000 |
| | FFRLE | retrovirus receptor 1 | | |
| 117 | FRRFVWN | Xenotropic and polytropic | 0.27500 | -0.09000 |
| | FFRLE | retrovirus receptor 1 homolog | | |
| 118 | FRRLPLRL | UDP-2,3-diacylglucosamine | 0.23083 | -0.20000 |
| | RLKI | hydrolase | | |
| 119 | FRRMHLR | Structure-specific endonuclease | 0.26833 | -0.07833 |
| | itffr | subunit SLX1 | | |
| 120 | FRSRLFYL | Exportin-T | 0.28000 | -0.11750 |
| | FHRF | | | |
| 121 | FRTFFRLP | Lycopene epsilon cyclase, | 0.31833 | -0.19667 |
| | KWMW | chloroplastic | | |
| 122 | FVFFFRW | Uncharacterized protein YBR090C | 0.22500 | -0.15750 |
| | RGNYK | | | |
| 123 | FVFKGRW | Matrix metalloproteinase-15 | 0.29750 | -0.19250 |
| | FWRVR | | | |
| 124 | FVIIMMW | Prolipoprotein diacylglyceryl | 0.17250 | -0.27667 |
| | RRKPK | transferase | | |
| 125 | FVIIMVW | Prolipoprotein diacylglyceryl | 0.17833 | -0.27500 |
| | RRKPR | transferase | | |
| 126 | FVIPRPRIP | ABC transporter G family member | 0.17583 | -0.32000 |
| | KWW | 29 | | |
| 127 | FWKRYH | Probable glucan 1,3-beta- | 0.28083 | -0.07500 |
| | KTFIFF | glucosidase D | | |
| 128 | FYFRPFRL | Membrane-associated protein Hem | 0.33083 | -0.11167 |
| | DWFR | | | |
| 129 | FYLIIRRK | Acetylcholine receptor subunit | 0.22667 | -0.28083 |
| | PLFY | delta | | |
| 130 | GGRWFR | Uncharacterized protein AF_2391 | 0.24667 | -0.15583 |
| | WFGRRF | | | |
| 131 | GHFIFKY | Oligopeptide transporter 6 | 0.26250 | -0.10167 |
| | RRVWW | | | |
| 132 | GLKYRLF | 4-alpha-L-fucosyltransferase | 0.28333 | -0.06250 |
| | YWLRR | | | |
| 133 | GYFVFWF | Fructose-like permease IIC | 0.19917 | -0.31333 |
| | RKVRL | component | | |
| 134 | IAMKLYF | Putative odorant receptor 83a | 0.18500 | -0.23500 |
| | RRFRP | | | |
| 135 | IFIKFRRP | 7-alpha-hydroxycholest-4-en-3-one | 0.19583 | -0.32333 |
| | DLLF | 12-alpha-hydroxylase | | |
| 136 | IFKFWLM | Glutamate decarboxylase 1 | 0.12583 | -0.35667 |
| | WKAKG | | | |
| 137 | IFKFWLM | Glutamate decarboxylase 1 | 0.12583 | -0.35667 |
| | WKAKG | | | |
| 138 | IFLKLIKF | Uncharacterized protein bbp_081 | 0.15667 | -0.36583 |
| | RIFQ | | | |
| 139 | IFRIFKLP | UPF0053 inner membrane protein | 0.12917 | -0.35917 |
| | MVRK | ytfL | | |
| 140 | IFSRYFIR | Putative adenosylcobalamin- | 0.28417 | -0.12083 |
| | RIRF | dependent ribonucleoside- | | |
| | | triphosphate reductase | | |
| 141 | IFYLIRFKI | Putative membrane protein ycf1 | 0.17917 | -0.40250 |
| | KLM | | | |
| 142 | IGGFFFLR | Uncharacterized endonuclease | 0.20167 | -0.31667 |
| | RFRR | C1958.04c | | |
| 143 | IILLLLVL | SLAM family member 6 | 0.13417 | -0.36500 |
| | RKRR | | | |
| 144 | IIRFRYFL | Sodium, potassium, lithium and | 0.23833 | -0.22917 |
| | RRLG | rubidium/H(+) antiporter | | |
| 145 | IKFWRMF | Uncharacterized | 0.26083 | -0.16833 |
| | FNLYK | flycosyltransferase MJ1069 | | |
| 146 | IKKYRYF | Maturase K | 0.29000 | -0.05750 |
| | FCHFW | | | |
| 147 | ILARPWR | Rhomboid family member 1 | 0.11750 | -0.40917 |
| | AFFKL | | | |
| 148 | ILFWKFY | GPI mannosyltransferase 4 | 0.30417 | -0.12000 |
| | RVHWK | | | |
| 149 | ILIVFIKK | UPF0118 membrane protein | 0.10750 | -0.39667 |
| | RIFK | HP_0567 | | |
| 150 | ILIVFIKK | UPF0118 membrane protein | 0.10750 | -0.39667 |
| | RIFK | jhp_0514 | | |
| 151 | ILLFFYPF | UPF0182 protein SUN_1015 | 0.22667 | -0.29000 |
| | YKKR | | | |
| 152 | ILLLIHFIL | Uncharacterized transporter | 0.14167 | -0.36667 |
| | KRR | YLL055W | | |
| 153 | ILPFKRRL | integral membrane protein GPR155 | 0.20167 | -0.24583 |
| | EFLW | | | |
| 154 | ILPYFLTR | Peroxisome biogenesis factor 10 | 0.18917 | -0.25333 |
| | LFRR | | | |
| 155 | ILRFRFFR | Mutator mutT protein | 0.27583 | -0.21417 |
| | CIKY | | | |
| 156 | ILRVIRLV | Potassium voltage-gated channel | 0.13917 | -0.36083 |
| | RVFR | subfamily A member 6 | | |
| 157 | IMWLFKM | Peroxisome assembly protein 12 | 0.17000 | -0.31833 |
| | KYARL | | | |
| 158 | IMYWVLK | ATP synthase subunit b | 0.19083 | -0.34083 |
| | KFLFK | | | |
| 159 | IPRPKIPV | Pleiotropic drug resistance protein | 0.21917 | -0.24417 |
| | WWRW | | | |
| 160 | IRFFLRLI | Undecaprenyl-diphosphatase | 0.20333 | -0.19667 |
| | NRVR | | | |
| 161 | IRRWRLR | tRNA(Ile)-lysidine synthase | 0.37500 | 0.11667 |
| | LYLHR | | | |
| 162 | IVMPLFLR | Uncharacterized protein HI_0976 | 0.17917 | -0.28000 |
| | RWKK | | | |
| 163 | IYGWRKR | Zeta-sarcoglycan | 0.22250 | -0.17417 |
| | CLYFF | | | |
| 164 | IYLKLLV | 60S ribosomal protein L18 | 0.14917 | -0.31417 |
| | KLYRF | | | |
| 165 | KFFFLRTR | Psychosine receptor | 0.21417 | -0.23000 |
| | RFAL | | | |
| 166 | KFKFPFR | Testis-specific Y-encoded-like | 0.27583 | -0.09417 |
| | RNPYF | protein 1 | | |
| 167 | KFLREPW | Putative uncharacterized protein | 0.26583 | -0.08167 |
| | CRHFF | YBL012C | | |
| 168 | KFLRFRR | Nucleoporin NDC1 | 0.19000 | -0.23250 |
| | SLLLL | | | |
| 169 | KFRFFYPI | 4-alpha-L-fucosyltransferase | 0.21583 | -0.24083 |
| | RRIA | | | |
| 170 | KFRLFYP | 4-alpha-L-fucosyltransferase | 0.18500 | -0.24167 |
| | LRRIA | | | |
| 171 | KFRTWRQ | Adenylosuccinate lyase | 0.33250 | 0.00083 |
| | LWLWL | | | |
| 172 | KFRYVW | Uncharacterized protein At3g49055 | 0.33250 | -0.11167 |
| | CWPMWR | | | |
| 173 | KFSRLRR | J domain-containing protein 1 | 0.35833 | -0.00667 |
| | FLWFR | | | |
| 174 | KIPLFMIK | Uncharacterized protein C3orf67 | 0.16000 | -0.29500 |
| | RKIW | homolog | | |
| 175 | KKFTYCF | Putative cyclic nucleotide-gated ion | 0.27083 | -0.20417 |
| | WWGLR | channel 13 | | |
| 176 | KLFFLVH | Maturase K | 0.19250 | -0.26417 |
| | YFVRR | | | |
| 177 | kLRWVRP | Glycyl-tRNA synthetase beta | 0.23583 | -0.13250 |
| | LRRIL | subunit | | |
| 178 | KLWLYKF | Uncharacterized mitochondrial | 0.32917 | -0.05583 |
| | IRRKF | protein 35 | | |
| 179 | KLYYFIR | Phosphate acyltransferase | 0.26750 | -0.09750 |
| | KIKMW | | | |
| 180 | KMWFVF | Aromatic-L-amino-acid | 0.14917 | -0.31583 |
| | RMYGIK | decarboxylase | | |
| 181 | KNFWRR | Protein crooked neck | 0.33667 | 0.01083 |
| | YIYLWI | | | |
| 182 | KRFAILR | tRNA(Ile)-lysidine synthase | 0.18333 | -0.25750 |
| | KWFCL | | | |
| 183 | KRFLLLFS | ATP-dependent RNA belicase has 1 | 0.18333 | -0.24500 |
| | FLKR | | | |
| 184 | KRHWLRF | Membralin | 0.33333 | 0.06000 |
| | FYLYH | | | |
| 185 | KRIFLLIFF | FMRFamide receptor | 0.29083 | -0.21917 |
| | KRR | | | |
| 186 | KRLRLLR | Probable multidrug resistance | 0.36333 | 0.12167 |
| | RWYRP | protein norM | | |
| 187 | KRPVFIFE | HEAT repeat-containing protein 5B | 0.20683 | -0.25000 |
| | WLRF | | | |
| 188 | KRPVFIFE | HEAT repeat-containing protein 5B | 0.20083 | -0.25000 |
| | WLRF | | | |
| 189 | KRRFYRLI | Maitrix protein | 0.29500 | -0.06667 |
| | MFRC | | | |
| 190 | KRSWWL | Phosphate acyltransferase | 0.31333 | -0.01750 |
| | LLLKRW | | | |
| 191 | KRSWWL | Phosphate acyltransferase | 0.31333 | -0.01750 |
| | LLLKRW | | | |
| 192 | KRSWWL | Phosphate acyltransferase | 0.31333 | -0.01750 |
| | LLLKRW | | | |
| 193 | KRSWWL | Phosphate acyltransferase | 0.31333 | -0.01750 |
| | LLLKRW | | | |
| 194 | KRSWWW | Phosphate acyltransferase | 0.39417 | 0.06250 |
| | LLLKRW | | | |
| 195 | KWWLCF | Probable actin-related protein 2/3 | 0.31500 | -0.15083 |
| | ARRRFM | complex subunit 3 | | |
| 196 | LAILKRR | Solute carrier family 35 member F2 | 0.26333 | -0.10750 |
| | WWKYM | | | |
| 197 | LARLLLY | Cytochrorne c biogenesis ATP- | 0.23333 | -0.17417 |
| | RRKLW | binding export protein CcmA | | |
| 198 | LARRRW | Probable potassium transport | 0.32417 | -0.02667 |
| | HWPWWA | system protein kup 1 | | |
| 199 | LFCWAW | Golgin subfamily A member 8-like | 0.28583 | -0.13750 |
| | LPRRRR | protein 2 | | |
| 200 | LFFKVFW | UPF0118 membrane protein | 0.33417 | -0.27833 |
| | RKFLR | TM_1349 | | |
| 201 | LFFRYRA | Exodeoxyribonuclease I | 0.24000 | -0.22250 |
| | RNFFI | | | |
| 202 | LFILKIFIR | Protein FPV175 | 0.13417 | -0.35333 |
| | RIN | | | |
| 203 | LFIRRPIL | ATP-dependent asparagine | 0.21083 | -0.27500 |
| | WMKK | adenylase 1 | | |
| 204 | LFLLGAIR | Protoheme IX farnesyltransferase | 0.13333 | -0.40083 |
| | IWRR | | | |
| 205 | LFLRIPFIR | Uncharacterized protein yqgO | 0.14917 | -0.33500 |
| | NKF | | | |
| 206 | LFLRYRA | Deoxyhypusine hydroxylase | 0.27167 | -0.22250 |
| | MFRLR | | | |
| 207 | LFQRRLL | Chromosome initiation inhibitor | 0.33417 | -0.10500 |
| | FWHRF | | | |
| 208 | LFQRRML | Chromosome initiation inhibitor | 0.32917 | -0.00833 |
| | YWHRF | | | |
| 209 | LFRKFRR | 7-alpha-hydroxycholest-4-en-3-one | 0.29667 | -0.17083 |
| | FDFLF | 12-alpha-hydroxylase | | |
| 210 | LFVVFFFR | Phosphatidylserine decarboxylase | 0.16750 | -0.32417 |
| | NPRR | beta chain | | |
| 211 | LGFLFYW | Putative B-type lectin protein L288 | 0.30333 | -0.05250 |
| | RHRYR | | | |
| 212 | LGIFRRC | Docking protein 6 | 0.11917 | -0.38417 |
| | WLVFK | | | |
| 213 | LILFWKF | ATP synthase subunit b | 0.19917 | -0.32333 |
| | VRPKY | | | |
| 214 | LILKKKM | DNA-directed RNA polymerase | 0.17417 | -0.31500 |
| | YIFYF | subunit beta′ | | |
| 215 | LIRFMLK | 3-ketoacyl-CoA synthase 12 | 0.12167 | -0.37917 |
| | LLIKK | | | |
| 216 | LIVRPFVF | Glutamate-ammonia-ligase | 0.12417 | -0.39500 |
| | RKYL | adenlyltransferase | | |
| 217 | LKAFFIRR | Uncharacterized | 0.20750 | -0.39083 |
| | YFVF | glycosyltransferase RP128 | | |
| 218 | LKAFFIRR | Uncharacterized | 0.20750 | -0.39083 |
| | YFVF | glycosyltransferase RT0209 | | |
| 219 | LKIFRRPR | Uncharacterized protein C12orf24 | 0.22417 | -0.20583 |
| | KLFM | | | |
| 220 | LKKFYRG | Maturase K | 0.27000 | -0.07833 |
| | RIWYF | | | |
| 221 | LIKRYAW | GRB2-associated-binding protein 1 | 0.31917 | 0.02667 |
| | KRRWFV | | | |
| 222 | LKRYAW | GRB2-associated-binding protein 1 | 0.31917 | 0.02667 |
| | KRRWFV | | | |
| 223 | LLAILRRR | Solute carrier family 35 member F1 | 0.30750 | -0.09750 |
| | WWKY | | | |
| 224 | LLFFFVM | Lectin-domain containing receptor | 0.19833 | -0.39667 |
| | YKKRL | kinase A4.2 | | |
| 225 | LLIIFPWR | Protein transport protein yif1 | 0.25917 | -0.20083 |
| | RRSW | | | |
| 226 | LLILLKYR | LEM domain-containing protein 2 | 0.24833 | -0.18917 |
| | WRKL | | | |
| 227 | LLKICRFF | Protein U52 | 0.23083 | -0.23000 |
| | NRFW | | | |
| 228 | LLMLIFLR | Choline transporter-like protein 4 | 0.17667 | -0.33083 |
| | QRIR | | | |
| 229 | LLPLLYY | Minor capsid protein L2 | 0.15833 | -0.29500 |
| | FLKKR | | | |
| 230 | LLPLRWL | Protein USP2 | 0.18911 | -0.38000 |
| | PLRRL | | | |
| 231 | LLQRRML | Chromosome initiation inhibitor | 0.29667 | -0.10917 |
| | FWHRF | | | |
| 232 | LLQRRML | Chromosome initiation inhibitor | 0.29667 | -0.10917 |
| | FWHRF | | | |
| 233 | LLRFLLR | Vitamin K-dependent gamma- | 0.20500 | -0.39083 |
| | KLYVF | carboxylase | | |
| 234 | LIRIVFRK | Maturase K | 0.21500 | -0.23167 |
| | RKIF | | | |
| 235 | LLVVVRL | GPI ethanolamine phosphate | 0.17833 | -0.31917 |
| | WLRRY | transferase 3 | | |
| 236 | LLWMPK | NADH-quinone oxidoreductase | 0.16250 | -0.31667 |
| | RLLKYI | subunit C/D | | |
| 237 | LMIILWK | Protein EV12B | 0.15583 | -0.32000 |
| | YLRKP | | | |
| 238 | LMKFFPF | THO complex subunit 2 | 0.19083 | -0.22333 |
| | EKRYF | | | |
| 239 | LMPWRW | Probable ubiquinone biosynthesis | 0.19000 | -0.30250 |
| | LPRKPL | protein ubiB | | |
| 240 | LMRIFRIL | Potassium voltage-gated channel | 0.14417 | -0.35083 |
| | KLAR | subfamily V member 2 | | |
| 241 | LPFPLRRL | Uncharacterized protein YJL147C | 0.18500 | -0.34667 |
| | LWRC | | | |
| 242 | LPRLFRFL | Ferrochelatase-2, chloroplastic | 0.15583 | -0.31167 |
| | QRPL | | | |
| 243 | LRFLFWK | Gamma-secretase subunit APH1- | 0.29167 | -0.18583 |
| | VYKRL | like | | |
| 244 | LRILPKIL | Acetylcholine receptor non-alpha 1 | 0.17417 | -0.33833 |
| | FMRR | chain | | |
| 245 | LRPAMRL | Mediator of RNA polymerase 11 | 0.15917 | -0.32333 |
| | RLRFI | transcription subunit 23 | | |
| 246 | LRRFLRF | Na(+)/H(+) antiporter subunit E | 0.29833 | -0.05500 |
| | DFYMR | | | |
| 247 | LRRFYRG | Maturase K | 0.32083 | -0.04917 |
| | WYL | | | |
| 248 | LRRFYRG | Maturase K | 0.32083 | -0.04917 |
| | RIWYL | | | |
| 249 | LRRIILLQ | Myosin-IXa | 0.30750 | -0.11583 |
| | RWFR | | | |
| 250 | IRRIVLLQ | Myosin-IXa | 0.27583 | -0.12083 |
| | RWFR | | | |
| 251 | LSFWGFK | ABC transporter G family member | 0.20250 | -0.22833 |
| | KIRWF | 6 | | |
| 252 | LVILKRK | Solute carrier family 35 member F2 | 0.24000 | -0.13750 |
| | WWKYI | | | |
| 253 | LWAFERI | Transmembrane protein 231 | 0.17083 | -0.26250 |
| | KRFVF | | | |
| 254 | LWFHFKR | Uncharacterized protein C19orf29 | 0.44500 | 0.21250 |
| | YRYRR | homolog | | |
| 255 | LWKMGF | Integrin alpha-9 | 0.29417 | -0.02750 |
| | FRRRYK | | | |
| 256 | LWLLFVP | Leucine-rich repeat and death | 0.15000 | -0.39250 |
| | PRVRR | domain-containint protein | | |
| 257 | LWWLRF | Putative membrane protein igaA | 0.28917 | -0.16833 |
| | RRPHPI | homolog | | |
| 258 | LWYFRKR | Undecaprenyl-diphosphatase 2 | 0.22167 | -0.21083 |
| | WCALV | | | |
| 259 | LYFFHKKI | Undecaprenyl-diphosphatase | 0.17417 | -0.27500 |
| | LR1L | | | |
| 260 | LYFRIRFY | Non-receptor tyrosine-protein | 0.38167 | -0.04083 |
| | FRNW | kinase TYK2 | | |
| 261 | LYLIYRKF | ATP synthase subunit b | 0.26833 | -0.17250 |
| | FFKK | | | |
| 262 | LYQRRML | Chromosome initiation inhibitor | 0.32917 | -0.00833 |
| | FWHRF | | | |
| 263 | LYRFFKRI | Na(+)/H(+) antiporter subunit A1 | 0.22000 | -0.17333 |
| | HLGW | | | |
| 264 | LYYLLRA | Regulator of telomere elongation | 0.17833 | -0.27583 |
| | MRRFV | helicase 1 homolog | | |
| 265 | MAAMRW | DnaJ homolog subfamily C | 0.26333 | -0.09167 |
| | RWWQRL | member 30 | | |
| 266 | MAFRWR | TM2 domain-containing protein 1 | 0.44917 | -0.12750 |
| | SLMRFR | | | |
| 267 | MAKLWF | WSC domain-containing protein 2 | 0.22417 | -0.17333 |
| | KFQRYF | | | |
| 268 | MALFRKF | Formin-like protein 7 | 0.14583 | -0.35750 |
| | FFKKP | | | |
| 269 | MALFRKF | Formin-like protein 6 | 0.18500 | -0.24417 |
| | FYRKP | | | |
| 270 | MARFFRR | 30S ribosomal protein S18 | 0.29083 | -0.02333 |
| | RKFCR | | | |
| 271 | MARFFRR | 30S ribosomal protein S18 | 0.29083 | -0.02333 |
| | RKFCR | | | |
| 272 | MARFFRR | 30S ribosomal protein S18 | 0.29083 | -0.02333 |
| | RKFCR | | | |
| 273 | MAWGW | Capsid protein | 0.36333 | 0.09833 |
| | WKRKRR | | | |
| | W | | | |
| 274 | MAWGW | Capsid protein | 0.48250 | 0.07000 |
| | WRRWRR | | | |
| 275 | MAWPWR | Capsid protein | 0.50167 | 0.13750 |
| | RRRWRW | | | |
| 276 | MAWWW | Capsid protein | 0.48250 | 0.07000 |
| | GRWRRR | | | |
| | W | | | |
| 277 | MAWYW | Capsid protein | 0.53917 | 0.29250 |
| | WRRRRRR | | | |
| 278 | MAWYW | ORF1/1 protein | 0.53917 | 0.29250 |
| | WRRRRRR | | | |
| 279 | MANVYW | ORF1/2 protein | 0.53917 | 0.29250 |
| | WRRRRRR | | | |
| 280 | MFFFFRF | Sulfhydryl oxidase 2 | 0.38333 | -0.13333 |
| | RSKRW | | | |
| 281 | MFFFWKK | Uncharacterized 66.5 kDa protein | 0.23417 | -0.16500 |
| | VKRIH | in trnJ-trnV interenic region | | |
| 282 | MFFKWIS | Uncharacterized 3.3 kDa protein in | 0.25083 | -0.16500 |
| | KFIRR | psbT-psbN intergenic region | | |
| 283 | MFFNFKK | Penicillin-sensitive transpeptidase | 0.17333 | -0.26083 |
| | YFLIK | | | |
| 284 | MFIFRGR | Collagenase 3 | 0.17083 | -0.39917 |
| | KFWAL | | | |
| 285 | MFYLIKK | Outer-membrane lipoprotein carrier | 0.10583 | -0.40833 |
| | LPKFI | protein | | |
| 286 | MIRIRNR | Protein srpA | 0.36583 | -0.02583 |
| | WFRWL | | | |
| 287 | MIYRRFK | Putative pterin-4-alpha- | 0.26750 | -0.09333 |
| | FRNFI | carbinolamine dehydratase | | |
| 288 | MIYRYLR | Dihydroorotate dehydrogenase | 0.27667 | -0.19500 |
| | PWLFK | | | |
| 289 | MKIWRFF | DNA-directed RNA polymerase | 0.24333 | -0.11500 |
| | LMKER | subunit bet″ | | |
| 290 | MKIYFWK | Putative uncharacterized protein | 0.30417 | -0.32417 |
| | LKFFF | DDB_G0268296 | | |
| 291 | MKKWRY | Maturase K | 0.30500 | 0.00333 |
| | YFVNFW | | | |
| 292 | MKLFWV | G-proteirt coupled receptor Mth | 0.21500 | -0.28750 |
| | KRLLR1 | | | |
| 293 | MKLLAFR | Probable ubiquinone biosynthesis | 0.15083 | -0.34750 |
| | RLLRI | protein ubiB | | |
| 294 | MKMILVR | Dentin matrix protein 4 | 0.14000 | -0.35250 |
| | RFRVL | | | |
| 295 | MKRRRR | Uncharacterized protein UL116 | 0.36167 | 0.10167 |
| | WRGWLL | | | |
| 296 | MKWLFK | UPF0161 protein Abu_1623 | 0.30583 | -0.21500 |
| | YLIRFY | | | |
| 297 | MKYLLIK | UPF0161 protein | 0.23500 | -0.32417 |
| | FVRFW | HY04AAS1_0880 | | |
| 298 | MLFYRFK | Cytochrome c oxidase assembly | 0.28583 | -0.08583 |
| | SWYRL | protein cox16, mitochondrial | | |
| 299 | MLIWWR | Probable branched-chain-amino- | 0.22667 | -0.18583 |
| | GKFRRA | acid aminotransferase | | |
| 300 | MLKFFLK | Uncharacterized protein US34A | 0.27250 | -0.08500 |
| | LRKRR | | | |
| 301 | MLKFLLK | Uncharacterized protein US34A | 0.27250 | -0.08500 |
| | FRKRR | | | |
| 302 | MLLKIKIK | Putative MSV199 domain- | 0.11500 | -0.38250 |
| | IRLF | containing 148R | | |
| 303 | MLLLRW | Cytochrome c-type biogenesis | 0.37250 | -0.31667 |
| | KRFWFL | protein CcmE | | |
| 304 | MLVLRKF | Pre-mRNA-splicing ATP- | 0.35250 | -0.06250 |
| | RWRKW | dependent RNA helicase PRP28 | | |
| 305 | MLWPFR | Putative adhesin P1-like protein | 0.42250 | -0.16167 |
| | WVWWKR | MPN_203 | | |
| 306 | MMFWRIF | Home exporter protein B | 0.28167 | -0.22333 |
| | RLELR | | | |
| 307 | MMKMAR | Testis anion transporter 1 | 0.14167 | -0.36000 |
| | FFYRLP | | | |
| 308 | MMPRLLF | Carnitine O-palmitoyltransferase 2, | 0.16083 | -0.36750 |
| | RAWPR | mitochondrial | | |
| 309 | MPRIFPW | Putative methionine | 0.26450 | -0.18417 |
| | KLWRK | aminoileptidase C | | |
| 310 | MPWWPW | Capsid protein | 0.56250 | 0.08833 |
| | RRWRRW | | | |
| 311 | MRFFKKY | Protein ycf2 | 0.30750 | -0.05250 |
| | LYYRI | | | |
| 312 | MRFLRWF | DNA dC->dU-editing enzyme | 0.38333 | 0.09583 |
| | HKWRQ | APOBEC-3G | | |
| 313 | MRFLRWI | Uncharacterized protein Corf61 | 0.38833 | -0.02917 |
| | RQIWR | homolog | | |
| 314 | MRFLSFR | Mannan-binding lectin serine | 0.21667 | -0.25000 |
| | RLLLY | protease 1 light chain | | |
| 315 | MRFVFFM | Protein dltB | 0.23500 | -0.27333 |
| | MKHKW | | | |
| 316 | MRIFRPW | Receptor-transporting protein | 0.22833 | -0.20167 |
| | RLRCP | | | |
| 317 | MRKWLY | Phosphatidylserine decarboxylase | 0.23250 | -0.15000 |
| | RLFIEL | beta chain | | |
| 318 | MRNRWI | Coiled-coil domain-containing | 0.33667 | -0.01500 |
| | WRFLRP | protein 90B, mitochondrial | | |
| 319 | MRSRWI | Coiled-coil domain-containing | 0.31583 | -0.04917 |
| | WRFLRP | protein 90B, mitochondrial | | |
| 320 | MRTLLIR | Protein N1 | 0.22417 | -0.18583 |
| | YILWR | | | |
| 321 | MRTLLIR | Protein N1 | 0.22417 | -0.18583 |
| | YILWR | | | |
| 322 | MRYFYV | Phosphoenolpyruvate carboxylase | 0.27583 | -0.20833 |
| | KWPFFK | | | |
| 323 | MSRFWHF | Defects in morphology protein 1, | 0.27250 | -0.06417 |
| | KKFYF | mitochondrial | | |
| 324 | MVFCLIL | T-lymphocyte activation antigen | 0.16000 | -0.33667 |
| | WKWKK | CD86 | | |
| 325 | MVLKFFR | Acyl-[acyl-carrier-protein] | 0.29083 | -0.40083 |
| | WLFRL | synthetase | | |
| 326 | MVLRRLL | UPF0454 protein C12or149 | 0.24000 | -0.13833 |
| | RKRWV | homolog | | |
| 327 | MVRILRW | UPF0161 protein A1S_2982 | 0.27917 | -0.29833 |
| | FIRLY | | | |
| 328 | MAIRILRW | UPF0161 protein AB57_0023 | 0.27917 | -0.29833 |
| | FIRLY | | | |
| 329 | MVRILRW | UPF0161 protein ABAYE3901 | 0.27917 | -0.29833 |
| | FIRLY | | | |
| 330 | MVRILRW | UPF0161 proteinABBFA_003529 | 0.27917 | -0.29833 |
| | FIRLY | | | |
| 331 | MVRILRW | UPF0161 protein ABSDF3681 | 0.27917 | -0.29833 |
| | FIRLY | | | |
| 332 | MVRILRW | UPF0161 protein ACICU_00008 | 0.27917 | -0.29833 |
| | FIRLY | | | |
| 333 | MVWFKR | Acetyl-coenzyme A carboxylase | 0.17833 | -0.28417 |
| | VKPFIR | carboxyl transferase subunit beta | | |
| 334 | MWCIRLR | IQ domain-containing protein F5 | 0.24500 | -0.26167 |
| | YLRLL | | | |
| 335 | MWFRNLI | Recombination-associated protein | 0.23833 | -0.13583 |
| | PYRIR | | | |
| 336 | MWKLWK | Light-harvesting protein | 0.21333 | -0.17667 |
| | FVDFRM | B800/830/1020 alpha-2 chain | | |
| 337 | MWRIRRR | IQ domain-containing protein F1 | 0.34500 | 0.02167 |
| | YCRLL | | | |
| 338 | MWRIWR | Light-harvesting protein B-870 | 0.26000 | -0.13500 |
| | LFDPMR | alpha chain | | |
| 339 | MWWWR | Capsid protein | 0.56083 | 0.13000 |
| | RRFWRPK | | | |
| 340 | MYFKKRR | CD48 antigen | 0.32333 | -0.17417 |
| | WFLIL | | | |
| 341 | MYKIFFR | Diydroorotate dehydrogenase | 0.24167 | -0.25833 |
| | LVFKR | | | |
| 342 | MYKLFFR | Dihydroorotate dehydrogenase | 0.24833 | -0.25500 |
| | LVFKR | | | |
| 343 | NILRILFW | PQ-loop repeat-containing protein 1 | 0.18667 | -0.24833 |
| | FGRR | | | |
| 344 | NLWKFW | EIB protein, small T-antigen | 0.32917 | 0.05000 |
| | LRRRVY | | | |
| 345 | PFMRWR | Ribulose bisphosphate carboxylase | 0.21917 | -0.18083 |
| | DRFLFC | large chain | | |
| 346 | PEMRWR | Ribulose bisphosphate carboxylase | 0.21917 | -0.18083 |
| | DRFLFC | lart.4e chain | | |
| 347 | PFMRWR | Ribulose bisphosphate carboxylase | 0.21917 | -0.18083 |
| | DRFLFC | large chain | | |
| 348 | PFMRWR | Ribulose bisphosphate carboxylase | 0.21917 | -0.18083 |
| | DRFLFC | large chain | | |
| 349 | PFMRWR | Ribulose bisphosphate carboxylase | 0.21917 | -0.18083 |
| | DRFLFC | large chain | | |
| 350 | PFMRWR | Ribulose bisphosphate carboxylase | 0.21917 | -0.18083 |
| | DRFLFC | large chain | | |
| 351 | PFMRWR | Ribulose bisphosphate carboxylase | 0.21917 | -0.18083 |
| | DRFLFC | large chain | | |
| 352 | PFMRWR | Ribulose bisphosphate carboxylase | 0.21917 | -0.18083 |
| | DRFLFC | large chain | | |
| 353 | PFMRWR | Ribulose bisphosphate carboxylase | 0.21917 | -0.18683 |
| | DRFLFC | large chain | | |
| 354 | PFMRWR | Ribulose bisphosphate carboxylase | 0.21917 | -0.18083 |
| | DRFLFC | large chain | | |
| 355 | PFMRWR | Ribulose bisphosphate carboxylase | 0.21917 | -0.18083 |
| | DRFLFC | large chain | | |
| 356 | PFMRWR | Ribulose bisphosphate carboxylase | 0.21917 | -0.18083 |
| | DRFLFC | larpe chain | | |
| 357 | PFMRWR | Ribulose bisphosphate carboxylase | 0.21917 | -0.18083 |
| | DRFLFC | large chain | | |
| 358 | PFMRWR | Ribulose bisphosphate carboxylase | 0.21917 | -0.18083 |
| | DRFLFC | large chain | | |
| 359 | PFMRWR | Ribulose bisphosphate carboxylase | 0.21917 | -0.18083 |
| | DRFLFC | large chain | | |
| 360 | PFMRWR | Ribulose bisphosphate carboxylase | 0.31083 | -0.06833 |
| | DRFLFR | large chain | | |
| 361 | PFMRWR | Ribulose bisphosphate carboxylase | 0.22250 | -0.21833 |
| | DRFLFR | large chain | | |
| 362 | PFMRWR | Ribulose bisphosphatc carboxylase | 0.22250 | -0.21833 |
| | DRFLFV | large chain | | |
| 363 | PERPWYF | Spore membrane assembly protein | 0.18667 | -0.48583 |
| | AMRLK | 1 | | |
| 364 | PIFIRRLH | Epstein-Barr nuclear antigen 3 | 0.15667 | -0.33917 |
| | RLLL | | | |
| 365 | PIFIRRLH | Epstein-Barr nuclear antigen 3 | 0.15667 | -0.33917 |
| | RLLL | | | |
| 366 | PLFIPYLR | Phospho-N-acetylmuramoyl- | 0.12083 | -0.38750 |
| | KLKF | pentapeptide-transferase | | |
| 367 | PLLAYRR | Putative DNA helicase Ino80 | 0.27167 | -0.09917 |
| | FWWKK | | | |
| 368 | PIRKLKV | DNA repair endonuclease UVH1 | 0.15083 | -0.30250 |
| | YFIFY | | | |
| 369 | PLWRLYR | Maturase K | 0.24667 | -0.11250 |
| | GRVWY | | | |
| 170 | QLKFRLF | 4-alpha-L-fucosylransferase | 0.30333 | -0.09667 |
| | YFLRR | | | |
| 371 | RALLRWF | Protein png-1 | 0.28667 | -0.13667 |
| | RRSFF | | | |
| 372 | RFFIPYLR | Phospo-N-acetylmuramoyl- | 0.24417 | -0.24917 |
| | KLKF | pentapeptide-transferase | | |
| 373 | RFKLFRM | tRNA(Ile)-lysidine synthase | 0.23167 | -0.27667 |
| | WLAKL | | | |
| 374 | RFKLLRM | tRNA(Ile)-lysidine synthase | 0.19417 | -0.28083 |
| | WLAKL | | | |
| 375 | RFLWKR | Uncharacterized protein MG316 | 0.32333 | 0.04167 |
| | WYLNKL | | | |
| 376 | RFLWLTL | Probale lysosomal cobalamin | 0.23500 | -0.17331 |
| | FKIRK | transporter | | |
| 377 | RFRLPFRR | Cathelicidin-3.4 | 0.24083 | -0.16417 |
| | PPIR | | | |
| 378 | RFRWRRR | Coiled-coil domain-containing | 0.32000 | -0.00583 |
| | LFVIS | protein 80 | | |
| 379 | RFYIRLIR | Isoleucyl-tRNA synthetase | 0.30750 | -0.03500 |
| | KRAW | | | |
| 380 | RFYMLLY | UPF0229 protein bll6755 | 0.23750 | -0.28333 |
| | VFLKR | | | |
| 381 | RGFKRLY | Ribosomal protein S7, | 0.28833 | -0.10583 |
| | FRFFK | mitochondrial | | |
| 382 | RGFRVLY | Neuronal-glial cell adhesion | 0.20167 | -0.21500 |
| | WRLGW | molecule | | |
| 383 | RIFIVQKIF | tRNA-specific 2-thiouridylase | 0.15500 | -0.30167 |
| | WIK | mnmA | | |
| 384 | RIFWYRH | Transmembrane and coiled-coil | 0.41333 | -0.05667 |
| | FRYFI | domain-containing protein 5B | | |
| 385 | RILRLFRR | Glutamate-ammonia-ligase | 0.34833 | -0.17333 |
| | RMMF | adenylyltransferase | | |
| 386 | RLFRRFRP | Lipoyl synthase | 0.34083 | -0.03500 |
| | RARF | | | |
| 387 | RLIRKFY | Putative membrane protein ycf1 | 0.30750 | -0.05917 |
| | YFLKY | | | |
| 388 | RLKMLVF | Putative transcription initiation | 0.22833 | -0.20917 |
| | RLIRR | factor TFIID 111 kDa subunit | | |
| 389 | RLRLLFW | Arginyl-tRNA synthetase | 0.20417 | -0.26000 |
| | VARFQ | | | |
| 390 | RPRIAVR | Heme A synthase | 0.20167 | -0.25833 |
| | RWLFL | | | |
| 391 | RQLFRFY | Menaquinone biosynthesis | 0.27917 | -0.13417 |
| | FKYIM | methyltransferase ubiE | | |
| 392 | RRIILLQR | Myosin-IXa | 0.26917 | -0.12417 |
| | WFRV | | | |
| 393 | RRIWWRF | Inner Membrane protein ybiR | 0.31583 | 0.02333 |
| | HLYSI | | | |
| 394 | RRKMMP | Putative mgpC-like protein | 0.30667 | -0.08750 |
| | RWWGWL | MPN_366 | | |
| 395 | RRWCPPP | Y-box-binding protein 2 | 0.26917 | -0.09250 |
| | FFYRR | | | |
| 396 | RVYLLRL | Innexin shaking-B | 0.22333 | -0.24500 |
| | RFRLV | | | |
| 397 | RWLLLQL | RNA-directed RNA polymerase L | 0.18167 | -0.27917 |
| | IKFVR | | | |
| 398 | RWMYLR | Large envelope protein | 0.35000 | -0.18917 |
| | RFIIYL | | | |
| 399 | RYRIPREI | Neutral and basic amino acid | 0.23417 | -0.13583 |
| | LFWL | transport protein rBAT | | |
| 400 | SFFRAFFR | Lycopene epsilon cyclase, | 0.15583 | -0.29167 |
| | VPKW | chloroplastic | | |
| 401 | SWKFRLF | 4-alpha-L-fucosyltransferase | 0.30333 | -0.05167 |
| | YLLAR | | | |
| 402 | TFFFAMM | Band 3 anion transport protein | 0.12000 | -0.37500 |
| | LRKFK | | | |
| 403 | TLIFFRKI | Uncharacterized membrane protein | 0.17167 | -0.32167 |
| | LWKI | bbp_130 | | |
| 404 | VFIRLFRR | Phospho-N-acetylmuramoyl- | 0.17750 | -0.25667 |
| | LQWG | pentapeptide-transferase | | |
| 405 | VFKNLYF | Menaquinone biosynthesis | 0.26250 | -0.13833 |
| | FYFRR | methyltransferase ubiE | | |
| 406 | VFKQLYF | Menaquinone biosynthesis | 0.24083 | -0.15833 |
| | FYFKR | methyltransferase ubiE | | |
| 407 | VFRLRFG | Probable DNA primase small | 0.20000 | -0.24667 |
| | YFIKR | subunit | | |
| 408 | VFRRFVW | Xenotropic and polytropic | 0.28417 | -0.25167 |
| | NFFRL | retrovirus receptor 1 | | |
| 409 | VFRRFVW | Xenotropic and polytropic | 0.28417 | -0.25167 |
| | NFFRL | retrovirus receptor 1 homolog | | |
| 410 | VFRRRRW | Helicase swr-1 | 0.32417 | -0.02250 |
| | HYMIL | | | |
| 411 | VFWVVW | Class II receptor tyrosine kinase | 0.26083 | -0.09167 |
| | RYRRRG | | | |
| 412 | VIRLVRV | Potassium-voltage-gated channel | 0.13250 | -0.37333 |
| | FRIFK | subfamily A member 5 | | |
| 413 | VLFRFRW | Uncharacterized protein MG242 | 0.27333 | -0.05833 |
| | KYIKH | homolog | | |
| 414 | VLIKRWP | Intraflagellar transport protein 122 | 0.14500 | -0.32083 |
| | PPLRW | homolog | | |
| 415 | VLLRVRM | Cbromodomain-helicase-DNA- | 0.17333 | -0.38000 |
| | LYFLR | binding protein 8 | | |
| 416 | VLPFYFI | Heme A synthase | 0.15583 | -0.39000 |
| | LRRK | | | |
| 417 | VRRRRTII | Probable G-protein coupled | 0.33417 | 0.06167 |
| | LRWW | receptor Mth-like 14 | | |
| 418 | VSFGRFL | UPF0761 membrane protein | 0.17833 | -0.28000 |
| | WRRFL | PXO_04555 | | |
| 419 | VSFGRFL | UPF0761 membrane protein | 0.17833 | -0.28000 |
| | WRRFL | XCV0968 | | |
| 420 | VSFGRFL | UPF0761 membrane protein | 0.17833 | -0.28000 |
| | WRRFL | XOO3417 | | |
| 421 | VSFGRFL | UPF0761 membrane protein | 0.17833 | -0.28000 |
| | WRRFL | XOO3615 | | |
| 422 | VVMTRIW | Probable potassium transport | 0.23917 | -0.15667 |
| | KWRLW | system protein kup 1 | | |
| 423 | VYFVIRLF | Uncharacterized protein C1B1.04c, | 0.19250 | -0.29500 |
| | RKYM | mitochondrial | | |
| 424 | VYLFRMR | Innexin shaking-B | 0.26083 | -0.23417 |
| | FRLVR | | | |
| 425 | VYLLRLR | Innexin shaking-B | 0.22333 | -0.24500 |
| | FRLVR | | | |
| 426 | WEYFRLR | Uncharacterized protein C19orfC1 | 0.33500 | 0.01667 |
| | PLRFR | | | |
| 427 | WFLYYRF | Golgi apparatus membrane protein | 0.39500 | 0.02417 |
| | KKRYL | TVP38 | | |
| 428 | WFYVFFY | G-protein coubled receptor | 0.35583 | -0.20583 |
| | RRLKL | homolog R33 | | |
| 429 | WIPERML | Lysosomal beta glucosidase | 0.24083 | -0.12583 |
| | RRYFL | | | |
| 430 | WIWACIR | DNA ligase 3 | 0.22000 | -0.17583 |
| | KRRLI | | | |
| 431 | WKCFFRR | Replication protein E1 | 0.31500 | -0.15750 |
| | LWARL | | | |
| 432 | WKFLRLY | Probable receptor-like protein | 0.26500 | -0.08583 |
| | FYPTR | kinase At5g38990 | | |
| 433 | WKILWFI | Probable palmitoyltransferase | 0.29583 | -0.18250 |
| | PFRQR | ZDHHC21 | | |
| 434 | WKILWFI | Probable palmitoyltransferase | 0.37333 | -0.18083 |
| | PFRRR | ZDHHC21 | | |
| 435 | WLIPYLR | Phospho-N-acetylmuramoyl- | 0.15833 | -0.30083 |
| | RLKFG | pentapeptide-transferase | | |
| 436 | WLIRIILR | DNA ligase 4 | 0.16917 | -0.27500 |
| | QMKL | | | |
| 437 | WLRRFLL | Protein ycf2 | 0.30750 | -0.08083 |
| | YRYLT | | | |
| 438 | WLYRFFF | Phosphate acyltransferase | 0.37167 | -0.15417 |
| | RFLQK | | | |
| 439 | WMYKYK | Uncharacterized protein C577.11 | 0.30167 | -0.01583 |
| | TPWFFR | | | |
| 440 | WRFAIFFL | Putative uncharacterized protein | 0.24500 | -0.27583 |
| | RTMR | YJL015C | | |
| 441 | WRRIRWA | Putative ABC transporter ATP- | 0.28667 | -0.06500 |
| | LKLVR | binding protein PH1815 | | |
| 442 | WWGWRR | Cobalamin synthase | 0.50500 | -0.00750 |
| | FLWRRL | | | |
| 443 | WWLWRT | Apolipoprotein N-acyltransferase | 0.31583 | -0.05667 |
| | ALAWRR | | | |
| 444 | YFRMRFY | Non-receptor tyrosine-protein | 0.37667 | 0.10000 |
| | FRNWH | kinase TYK2 | | |
| 445 | YIFFRYHR | Ribosome production factor 1 | 0.32750 | -0.04917 |
| | YLFK | | | |
| 446 | YIFIKKKG | Protein ycf2 | 0.21167 | -0.33250 |
| | WFFF | | | |
| 447 | YKFWLRT | Zinc finger protein C1039.05c | 0.30000 | -0.09750 |
| | YRVFF | | | |
| 448 | YLALYRR | Uncharacterized protein BALF 1 | 0.23167 | -0.19917 |
| | LWFAR | | | |
| 449 | YMINVRW | NADH-quinone oxidoreductase | 0.27500 | -0.09917 |
| | TIPRFR | subunit H | | |
| 450 | YMWVRW | NADH-quinone oxidoreductase | 0.27500 | -0.09917 |
| | TIPRFR | subunit H | | |
| 451 | YQRMMY | Evolutionarily conserved signaling | 0.29333 | -0.04083 |
| | WFPRFK | intermediate in Toll pathway, | | |
| | | mitochondrial | | |
| 452 | YVFLYWR | Alpha-1,2 glucosyltransferase | 0.24508 | -0.20667 |
| | RLLKP | ALG10 | | |
| 453 | YWPKRA | Uncharacterized protein C1orf161 | 0.28000 | -0.07500 |
| | RWPRLF | homolog | | |
| 454 | YIATRRFW | Undecaprenyl-diphosphatase | 0.30333 | -0.03833 |
| | WINSPK | | | |
| 455 | YYIFRRFK | Oligopeplide transporter 1 | 0.30917 | -0.02167 |
| | TWWA | | | |

1. A peptide wherein the PP1 of the peptide is <[(the PP2 of the peptide*X1)+X], wherein X1 is 1.7 to 2.3 and X is −0.6 to −0.85.

2. The peptide of claim 1, selected from the group consisting of SEQ ID NOs. 1-455.

3. The peptide of claim 2, selected from the group consisting of SEQ ID NOs. 1-9.

4. The peptide of claim 2 selected from the group consisting of SEQ ID NOs. 10, 11, 15, 16, 17 and 18.

5. A peptide of any of claims 1 to 4, which is conjugated to a small molecule, nucleic acid, peptide or protein.

6. A method of identifying cell penetrating peptides among a group of peptides by: (1) determining the PP1 of said peptides; (2) determining the PP2 of said peptides; (3) identifying peptides within the group, wherein PP1<[(PP2*X1)+X], wherein X1 is 1.5 to 10 and X is 0.3 to −1.5; and (4) testing the peptides identified in step 3 in an in vitro or in vivo assay to confirm that said peptides are cell-penetrating.

7. A method for the treatment of cancer or a virological, central nervous system, inflammatory, immune, or metabolic disease or condition, comprising administering to a patient in need thereof, a therapeutically effective amount of a peptide according to any one of claims 1 to 5.

8. An isolated nucleotide encoding a peptide according to any of claims 1 to 5.

9. A vector comprising an isolated nucleotide according to claim 8.

10. Use of a peptide according to any one of claims 1 to 5 for the treatment or prophylaxis of cancer or a virological, central nervous system, inflammatory, immune, or metabolic disease or condition.

11. The invention hereinbefore described.
